(19) United States
(12) Patent Application Publication
(10) Pub. No.: US 2026/0102568 A1
(43) Pub. Date: Apr. 16, 2026

(54) USER-TRIGGERED RECONSTITUTION ADMINISTRATION DEVICE

(71) Applicant: AKTIVAX, INC., Broomfield, CO (US)

(72) Inventor: Amir Genosar, Broomfield, CO (US)

(21) Appl. No.: 19/115,169

(22) PCT Filed: Apr. 6, 2023
(86) PCT No.: PCT/US2023/017754

§ 371 Date: Mar. 25, 2025

Related U.S. Application Data
(60) Provisional application No. 63/328,150, filed on Apr. 6, 2022.
(60) Provisional application No. 63/376,853, filed on Sep. 23, 2022.

(51) Int. Cl. A61M 5/24; A61M 5/20

(52) U.S. Cl. CPC A61M 5/2448; A61M 5/2066; A61M 5/2459; A61M 2005/202; A61M 2005/2073; A61M 2005/2451

(57) ABSTRACT

An administration device incorporating a reconstitution mechanism which may be operated by a user to combine a first compartment and second compartment. The reconstitution mechanism may include a roller arranged such that actuation of a trigger by the user causes the roller to move in a distal direction, the distal movement being such that the roller may compress a first compartment and to establish fluid communication with a second compartment. The compartments may be included in a flexible package and separated by a frangible seal. Contents of the compartments may mix, as in the case of reconstitution of a beneficial agent in a diluent. A visual indicator of the usability of a mixed substance according to conditions of time and/or temperature may be provided.

### CROSS-REFERENCE TO RELATED APPLICATIONS

This application claims benefit of priority to the prior-filed U.S. patent application Ser. No. 63/328,150 entitled “User Triggered Drug Reconstitution Mechanism” filed on Apr. 6, 2022, and to prior-filed U.S. patent application Ser. No. 63/376,853 entitled “Reconstitution Drug Delivery Device with Integrated Visual Time Indicator” filed on Sep. 23, 2022. The teachings of the priority patent applications are incorporated by reference in their entirety. In the event of a conflict between the use or definition of any term in the present application and its use or definition in an application incorporated by reference, the use or definition in the present application controls.

### TECHNICAL FIELD

The present application relates to beneficial agent administration devices and, more particularly, to beneficial agent administration devices that operate on prefilled beneficial agent prefilled storage assemblies.

### BACKGROUND

Autoinjectors are self-contained, prefilled drug delivery systems designed to administer a fixed parenteral drug dose at predetermined injection depth and speed. They automate needle insertion and drug injection, enabling rapid intramuscular (IM) delivery and self-administration with minimal training. For this reason, autoinjectors are the preferred systems for administering emergency life-saving treatments, including medical countermeasures (MCMs) against chemical threats. Some of the limitations of current autoinjector technology stem from the challenges of implementing reconstitution capability. In reconstitution auto-injectors, the device can contain a drug separated into dry and wet components and combine them for delivery at some point prior to injection.

### SUMMARY OF THE DISCLOSURE

We present an administration device including a merging mechanism, which may be configured to merge a first and second compartments of a storage assembly. This can allow for the storage of a beneficial agent separated into wet and dry components and for the reconstitution of these components at some point prior to activation and administration. This may be desirable for improved drug stability, which can translate to longer shelf-life, storage without refrigeration, ability to withstand longer excursions in field conditions, accommodating molecules that are unstable in solutions like biopharmaceuticals such as butyrylcholinesterase, and accommodating long-acting, slow-release micro-particle formulations. The storage assembly may comprise high-barrier film formed into adjacent compartments of a film package wherein a dry active ingredient and a diluent may be stored. The compartments may be separated by a frangible seal. Such primary drug containers may demonstrate equivalent or improved performance relative to glass vials when evaluated for moisture ingress. Additionally, we present an indicator configured to produce a signal to communicate information related to a stored and/or reconstituted beneficial agent. Such an indicator may be activated in the process of reconstitution to convey information related to the stability and safety of the mixed/reconstituted substance.

We recognize that reconstitution within a prefilled drug delivery system presents two major problems. First, reconstitution may not be instantaneous due to the poor solubility of the substances and/or limitations of the reconstitution mechanism of the device. The user may then be required to wait a certain amount of time before the beneficial agent can be administered. Second, beneficial agents are often formulated for reconstitution as they are unstable in solutions and degrade in relatively short timeframes, from minutes to days. This leads to the loss of their potency and, in turn, the treatment becoming ineffective and potentially harmful if the degradation products are toxic. Thus, when considering the current methods and reagents used for reconstitution, the user must administer prefilled reconstitution products within a specified time window, which requires tracking time from the moment of reconstitution. This introduces a risk of user errors. Ambient temperature may influence the beneficial agent degradation rate, either extending or shortening the time window for administration.

In the specific case of prefilled emergency-use reconstitution autoinjectors that are used to treat life-threatening conditions like anaphylaxis, seizures, and nerve agent poisoning, the consequences of failure to use the autoinjector as intended are likely to be catastrophic. Furthermore, situations that warrant the use of such autoinjectors are often chaotic and extremely stressful and take place in challenging or even austere environments. A time-tracking device may not be accessible, and the users'ability to perform relatively simple mental tasks is often compromised.

Throughout this disclosure, the terms “proximal” and “distal” are used to orient the administration device and the configuration of the device. As used herein, the term “proximal” generally refers to a direction towards the base or dispensing end of the administration device. As used herein, the term “distal” refers to a direction towards the end furthest from the base or dispensing end of the administration device.

One mode of the present invention relates to a reconstitution autoinjector incorporating the invention, which may be employed for medical countermeasure (MCM) applications under battlefield conditions, and which may overcome the limitations of other devices. Moreover, this disclosure describes the addition of a visual indicator to prefilled administration devices, such as autoinjectors, that provide means by which to measure a desired temporal or environmental metric. The primary drug container (PDC) may be glass-free, flexible, and constructed from a high-barrier film, resulting in a single drug contact material. The wet and dry compartments of the PDC are separated by a material-bonded seal rather than a movable plunger, eliminating the known issue of moisture migration. However, rigid vials utilizing a movable plunger may be incorporated as an alternative configuration. The reconstitution process may be fully automatic and may be initiated by rotating a merging trigger (e.g., a cap or a knob of the administration device) which can be disposed at the distal end of the device. Reconstitution may be accomplished together with the arming of the device by the user to expel a medical agent. However, the device can also be configured to allow the user to perform reconstitution without arming the device. This alternative allows the reconstituted agent to maintain its sterility in a stored state substantially without the risk of unintentional dispensing, which would be present in a device stored in the armed state. Administration of the drug may be performed in some versions by depressing the proximal end of the autoinjector against the target injection site. Upon activation, compressed carbon dioxide or an equivalent gas may be released from a pressurized fluid source such as a canister to drive a sequence of full-depth needle deployment prior to initiation of drug injection, drug injection, and needle retraction after a preset dwell time.

The combination of pressure activation and the use of a flexible PDC may eliminate the mechanical constraints of rigid PDC autoinjectors and hence enable a range of delivery volumes based on the same device components and sub-assemblies. There do not need to be any moving parts in the primary container, which can enable a more reliable reconstitution approach. Avoidance of glass or a syringe-like form factor may present a number of benefits, including eliminating the risk of a harmful interaction between silicone lubricants and rubber leachables with the drug. In addition, a reconstitution auto-injector may accommodate two reconstitution PDCs and perform dual-reconstitution and dual-injection through two drug-exclusive needles. The reconstitution mechanism described herein may be adaptable to other administration forms, such as medical agent delivery devices for otic, intranasal, oral, or rectal administration.

Administration devices implementing the teachings of this disclosure, including those comprising an auto-injector, may be configured to reconstitute a dry formulation of 1.67 mg atropine base in 0.7 mL to 2.0 mL diluent volume and further configured for subsequent intramuscular administration. Alternative embodiments may employ other drug formulations, such as a naloxone formulation comprising a flowable powder of microparticles 50 μm-100 μm in size. A single dose of 200 mg of powder may be reconstituted in a 1 mL volume of diluent.

Some versions of the administration device may support self-administration or administration by medically trained and/or non-medically trained persons in austere operational settings and may be manufactured to maintain full functionality after prolonged exposure to austere environmental conditions. View of the merging/reconstitution process may not be visible to the user in every variation of the administration device. This can be done in cases where additional user actions like shaking are not required to fully mix/reconstitute the drug product. Such variations may be operable under conditions of reduced visibility and user dexterity. Other variations may include a visible mark, scratch, toggle, or some colored or otherwise labeled indicia or signal to clearly indicate whether the drug container within the administration device is in the reconstituted state. The administration device may employ a reconstitution formulation of a medical agent to improve reconstitution quality. The primary drug container may employ existing or emerging film materials or composite webs compatible with the desired beneficial agent formulation and frangible seals. Merging of the first and second compartments of the storage assembly may be triggered independently of or together with the arming of the administration device. Such merging and/or arming may be triggered by user manipulation of a merging trigger, such as by removal of a cap, twisting of a cap or knob, pressing of a lever or button, or by any such user manipulation of a merging and/or arming trigger.

A storage assembly comprising a dual-compartment flexible primary drug container may be divided by a frangible seal into a proximal dry compartment and a distal wet compartment. Reconstitution functionality may be enabled by a spring-loaded carriage, including a compression element that moves relative to the storage assembly when released into a position wherein it may squeeze and depresses a compartment of the primary container to break the frangible seal and allow the wet and dry compartments to merge. The drug container geometry may be optimized to minimize stresses and ensure thorough mixing.

The device may incorporate a lanyard of such shape and material, labeling, and feel of arming step as required for human use. The present merging mechanism design may likewise be adaptable to variations that suit user needs. In one alternative, an arming mechanism that separates reconstitution and arming into discrete steps may be employed. In variations incorporating or permitting a two-step administration approach, the first step may comprise removing a cap to arm the device, which may also trigger the reconstitution mechanism to merge the wet and dry compartments. In embodiments incorporating three-step operation, twisting a cap or knob or otherwise actuating a merging trigger may be the first discrete step causing the merging of two containers of the storage assembly without activating the administration device or arming the device for administration. Arming and administration may then be carried out as with a two-step operation. Variation of specific implementation modalities may allow for the invention to be adapted to a wide variety of drug combinations with varying reconstitution characteristics. Triggering the mixing of the two substances without removing the cap may further allow a user to safely prepare the administration device in advance of an anticipated use without the risk of accidental activation inherent to waiting with an armed device. In some variants, the separate merging step may serve to alert the user that an administration device is a reconstitution type by not permitting cap removal or arming without the reconstitution action being performed first. The three-step operation may be accomplished by manipulating a merging trigger, such as by a 90-degree or 180-degree rotation of a cap, end-piece, or knob, or by pressing a lever. Device packaging may incorporate a label with detailed required information and guiding user operation.

Because the reconstitution of a beneficial agent may not be immediate, situations may arise where the beneficial agent is reconstituted prior to use or anticipated need. However, drugs differ in their reconstitution kinetics and in their stability once dissolved, affecting the amount of time required to reach complete reconstitution and the amount of time they can spend in solution before they appreciably lose potency. Furthermore, the times may vary with ambient temperature. Therefore, there is great utility in the use of a visual indicator that is automatically actuated upon initiating the reconstitution process to inform the user, by visual cues, of the time window when the drug can be administered safely and effectively. A visual indicator is therefore calibrated for each individual drug and may be configured to track the effect of time and temperature. By observing a change in color, shape, pattern, writing on the visual indicator, or a combination thereof, the user is informed regarding the reconstitution state and if the drug is ready to be administered. The user can also determine whether a reconstituted drug has passed its window of administration and must be discarded.

A visual indicator may be attached to the body of the autoinjector, may be integrated with the body, or may be disposed inside the body. The reconstitution process may be initiated by rotating a cap or a knob, pressing a lever, or otherwise actuating a trigger on the device. This may further actuate the visual indicator by transferring a force to the visual indicator to actuate it by pressing, pulling, peeling, or a combination thereof. These actions serve to change the physical configuration of the indicator where a chemical, physical, enzymatic, or biological process is initiated and consequently tracks time or time associated with temperature. This process may be irreversible. Moreover, the visual indication can be in binary “Use Now”/“Do Not Use” form or can indicate the current point within the administration time window. The visual indication may rely on the color change as the means of changing appearance and communicating information to the user.

The visual indicator may continuously track time alone, on the order of seconds, minutes, hours, days, or weeks. The indicator may also track time only when the prefilled device is experiencing a specific temperature range. The indicator may also track the combined effect of time and temperature measured by Mean Kinetic Temperature (MKT). The indicator is configured such that the timing of the information it conveys matches the reconstitution and lifespan profile of the beneficial agent.

In some configurations, once activated, the visual indicator first changes in appearance to indicate when the reconstitution process has been completed and the beneficial agent is ready to be administered. At a later time that is either predefined or influenced by ambient temperature, the visual indicator undergoes additional changes in appearance to indicate that the beneficial agent has expired and should no longer be administered.

### BRIEF DESCRIPTION OF THE DRAWINGS

FIG. 1a illustrates a general view of an auto-injector with a rotatable end piece.

FIG. 1b illustrates a general view of an auto-injector with a rotatable end piece in a pre-reconstitution state with the shell sectioned.

FIG. 1c illustrates a general view of an auto-injector with a rotatable end piece in a post-reconstitution state with the shell sectioned.

FIG. 1d illustrates a section view of an auto-injector with a rotatable end piece in a pre-reconstitution state.

FIG. 1e illustrates a section view of an auto-injector with a rotatable end piece in a pre-reconstitution state showing a pressure source and activation system.

FIG. 1f illustrates a section view of an auto-injector with a rotatable end piece in a post-reconstitution state.

FIG. 1g illustrates a section view of an auto-injector with a rotatable end piece in a post-reconstitution state showing a pressure source and activation system.

FIG. 2a illustrates a general view of a primary drug container.

FIG. 2b illustrates a section view of a primary drug container.

FIG. 3 illustrates a general view of a carriage assembly.

FIG. 4a illustrates a section view of an auto-injector with a rotatable body and shell in a pre-reconstitution state.

FIG. 4b illustrates a section view of an auto-injector with a rotatable body and shell in a pre-reconstitution state showing a pressure source and activation system.

FIG. 5a illustrates a general exterior view of an auto-injector.

FIG. 5b illustrates a general internal view of an auto-injector in a pre-reconstitution state.

FIG. 5c illustrates a general internal view of an auto-injector in a post-reconstitution state.

FIG. 6a illustrates a general exterior view of an auto-injector.

FIG. 6b illustrates a section view of an auto-injector in a pre-reconstitution state.

FIG. 6c illustrates a section view of an auto-injector in a pre-reconstitution state showing a pressure source and activation system.

FIG. 6d illustrates a close-in view of a retaining element and compression element.

FIG. 6e illustrates a section view of an auto-injector in a post-reconstitution state.

FIG. 6f illustrates a section view of an auto-injector in a post-reconstitution state showing a pressure source and activation system.

FIG. 7 illustrates a section view of an auto-injector including more than one primary drug container and arranged in a pre-reconstitution state.

FIG. 8a illustrates a general view of an auto-injector in a pre-reconstitution phase with an indicator including an actuating arm mounted to a cap.

FIG. 8b illustrates a section view of an auto-injector in a pre-reconstitution phase with an indicator including an actuating arm mounted to a cap.

FIG. 8c illustrates a section view of an auto-injector in a post-reconstitution phase with an indicator including an actuating arm mounted to a cap.

FIG. 9a illustrates a general view of an auto-injector in a pre-reconstitution phase with an indicator including an actuating strip mounted to a cap.

FIG. 9b illustrates a section view of an auto-injector in a pre-reconstitution phase with an indicator including an actuating strip mounted to a cap.

FIG. 9c illustrates a section view of an auto-injector in a post-reconstitution phase with an indicator including an actuating strip mounted to a cap.

FIG. 10a illustrates a general view of an auto-injector in a pre-reconstitution phase with an indicator including an actuating arm mounted to a body.

FIG. 10b illustrates a section view of an auto-injector in a post-reconstitution phase with an indicator including an actuating arm mounted to a body.

FIG. 11a illustrates a pressure-actuated indicator in a pre-actuated state

FIG. 11b illustrates a pressure-actuated indicator in a post-actuated, safe and ready to use state

FIG. 11c illustrates a pressure-actuated indicator in a post-actuated, unfit to use state

FIG. 12a illustrates a pull-actuated indicator in a pre-actuated state

FIG. 12b illustrates a pull-actuated indicator signaling a safe and ready to use state.

FIG. 12c illustrates a pull-actuated indicator signaling an unfit to use state.

FIG. 13a illustrates a peel-actuated indicator in a pre-actuated state

FIG. 13b illustrates a peel-actuated indicator signaling a safe and ready to use state

### DETAILED DESCRIPTION OF THE INVENTION

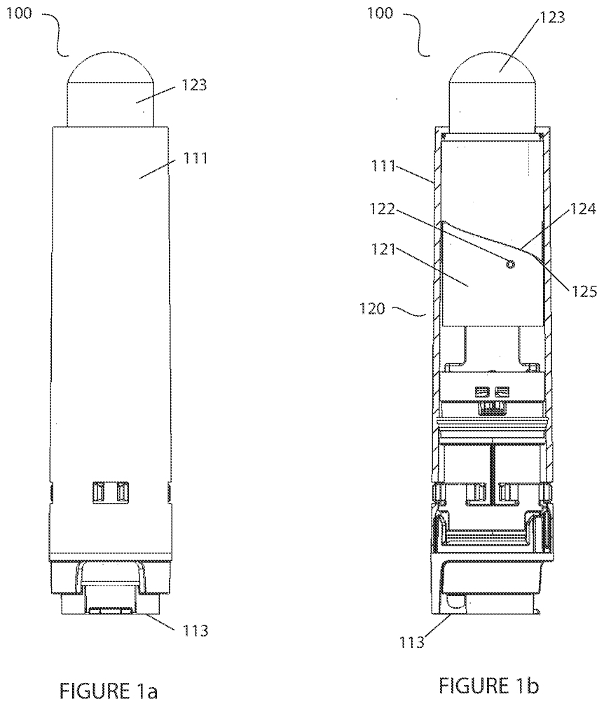

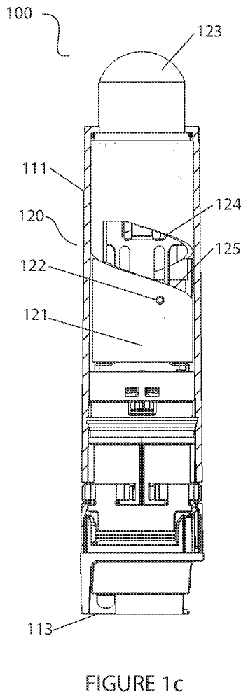

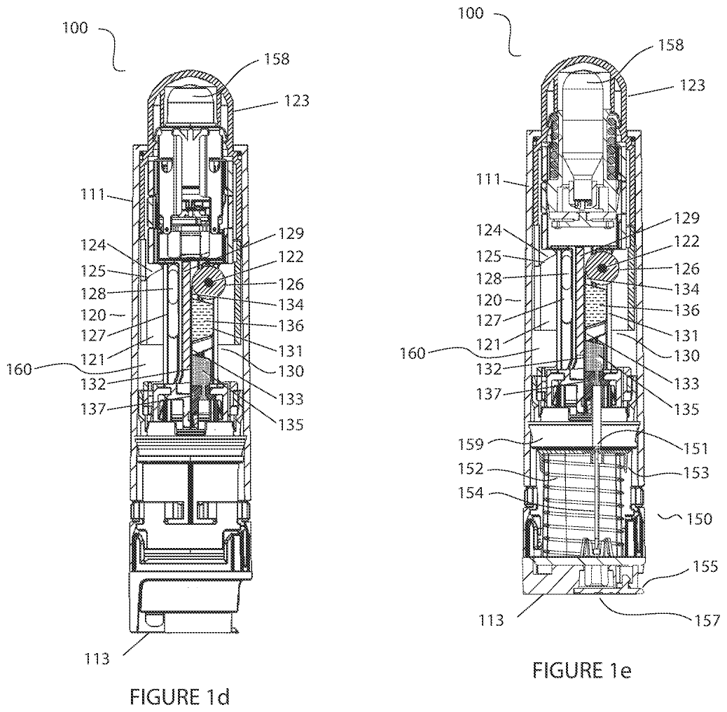

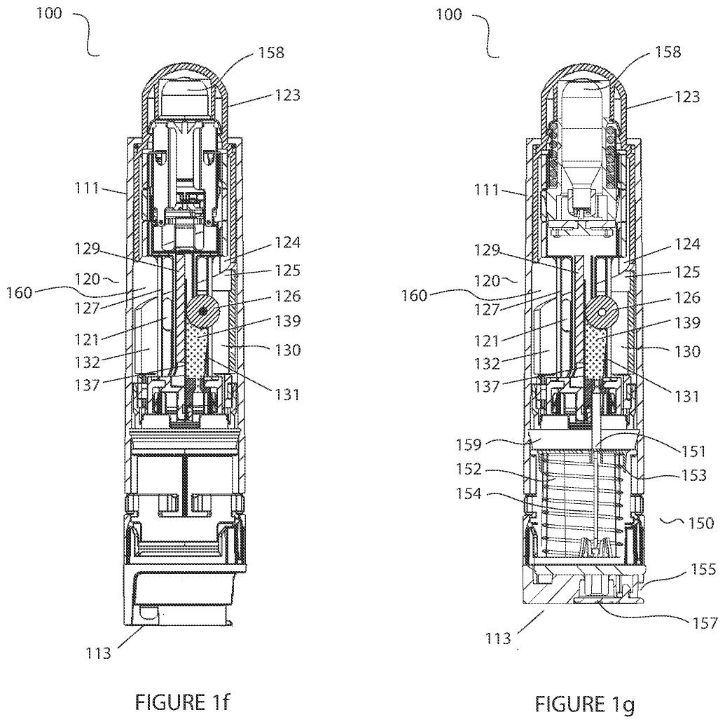

FIGS. 1a-c illustrate an administration device 100 comprising an auto-injector. The administration device 100 may include a merging mechanism 120, which may be configured for manual actuation by manipulation of a merging trigger 123, including a second interface element 124 which may be a cam surface. Some variations may include a visible mark, scratch, toggle, or some colored or otherwise labeled indicia visible through or on the shell 111 to clearly indicate whether the storage assembly 130, here illustrated as a primary drug container formed from a flexible material, has transitioned from a first state in which the stored substances (e.g., contents) are stored separately in a first compartment 136 and a second compartment 135 to a second state wherein the stored substances are mixed/reconstituted by merging the first compartment 136 and the second compartment 135 to create a reconstituted interior volume. The stored substances may include a first constituent (e.g., a diluent) and a second constituent (e.g., an active ingredient) that may be mixable to form a deliverable beneficial agent. Further compartments and further constituents (e.g., different from or the same as the first and second constituents) for combination are contemplated. The administration device 100 may be configured to reconstitute when mechanical manipulation is applied to a merging trigger 123, which may include a knob, lever, or button and may be disposed at the distal end. The manipulation may include rotating or actuating linear motion of the merging trigger 123. The manipulation may include a rotation or linear motion of a first portion (e.g., a cap or knob) of the administration device 100 relative to a second portion (e.g., a body or the shell 111) of the administration device 100. The merging trigger 123 may further be used (e.g., by the same or different manipulations) to transition the activation system 150 from a safe state to an armed state and/or to activate the activation system (e.g., to administer the beneficial agent from the administration device 100 to a patient).

An administration device 100 may comprise a shell 111, a distal end comprising a merging trigger 123, and a proximal end 113 (e.g., an administration end from which a beneficial agent is administered. A carriage 121 may comprise a first interface element 125, such as the illustrated cam surface. The carriage 121 may further comprise a compression element with a cross-member 122. The merging trigger 123 may comprise a second interface element 124, such as the illustrated cam follower. When included, the cam surface and cam follower may be relatively disposed such that proximal translation of the carriage 121 occurs when the merging trigger 123 is actuated. This is demonstrated in the rotation of the merging trigger 123 (shown in the form of a knob) and the translation of the carriage 121 between FIGS. 1b & 1c, with FIG. 1b depicting the pre-translation state and FIG. 1c depicting the post-translation state.

FIGS. 1d-g illustrate cross-section views of an administration device 100 both before and after merging of the compartments of the storage assembly 130. The merging mechanism 120 may include a carriage 121 with a cross-member 122 and a compression element 126. The compression element 126 may be any of a bar, roller, rod, press, or an equivalent. The carriage 121 may further comprise a first interface element 125 disposed against a second interface element 124 of the merging trigger 123, and the compression element 126 may be supportably engaged to the first interface element 125. A follower 128 may extend into a guide slot 127 disposed against a rigid backing member 129. The storage assembly 130 may comprise a formed side 131, which may be composed of a formed polymer providing a high barrier to fluid ingress. The storage assembly 130 may further comprise a lid side 132 which may be of the same or different material, such as an aluminum-backed polymer surface. When the storage assembly is in an initial state, a fluid isolation element 133 such as a frangible seal may be arranged to prevent fluid communication between (e.g., fluidly isolate) a second compartment 135, which may contain a beneficial agent in dry form, from a first compartment 136, which may contain a diluent such as water or a saline solution. The frangible seal, first compartment 136, and second compartment 135 may be formed by sealing a first wall against a second wall. The frangible seal between the first wall and the second wall may be weaker than the seal between the first wall and the second wall defining the first compartment 136 and the second compartment 135. A non-frangible 134 may provide a durable barrier that is not normally breached during reconstitution or dosage administration. A fitment 137 may be included and may be configured to maintain the storage assembly 130 in place within the administration device 100. The storage assembly 130 may include a port from which the beneficial agent may be expelled.

The second interface element 124 may comprise a cam surface, and the first interface element 125 may comprise a cam follower, and these elements may comprise opposing inclined surfaces arranged such that relative rotation between the opposing inclined surfaces may result in the linear movement of one of the interface elements and any structure supportably engaged thereto. Rotational movement of the merging trigger 123 may move the second interface element 124 to engage against the first interface element 125 to cause linear motion of the carriage 121. The compression element 126 may move relative to the storage assembly 130 to a second position wherein it may be disposed for contact of at least one of the first compartment 136 or the second compartment 135, defeating the fluid isolation element 133 and merging the compartments to permit fluid communication between their respective interior volumes. In implementations, the carriage 121 is moveable with the compression element 126. A merged volume 139 can be made to maintain a mixed substance, including a reconstituted medical agent, in a stored and sterile state.

Manipulation of an arming trigger may manipulate an arming system engageable with an activation system 150 of the administration device 100. Manipulation of the merging trigger may optionally be independent of the action of the arming system, although the merging trigger may comprise the arming trigger in some variations. The arming system may be configured to transition the activation system 150 between a safe state in which the activation system 150 is configured to prevent activation of the administration device 100 for dispensing the beneficial agent and an armed state in which the activation system 150 is configured to allow activation of the administration device 100 for dispensing the beneficial agent. The activation system 150 may include a pressurized fluid source 158, such as a canister containing a pressurized gas or liquid. In transitioning the activation system from the safe state to the armed state, the arming system may actuate the pressurized fluid source 158 to a position confronting a fluid source activator such as a rupturing pin. The pressure chamber 160 may be configured to receive the pressurized fluid upon release of the pressurized fluid. The configuration of the armed position may be such that the administration device 100 will activate and administer the beneficial agent to a target site when the proximal end 113 is pushed against the target site. In an implementation, the arming trigger resists actuation when the administration device 100 is in an initial state in which the first compartment 136 and the second compartment 135 are unmerged, and the arming trigger is free to actuate (e.g., released by the action of the merging system) to arm (e.g., a transition from a safe to an armed state) the activation system 150 when the first compartment 136 and the second compartment 135 are merged in a merged state.

The merging trigger may comprise an external portion that is external to the pressure chamber 160 and an internal portion disposed within the pressure chamber 160, the internal portion further comprising the second interface element. In an implementation, the merging trigger 123 is configured to transfer a motion from the exterior (e.g., outside) of the pressure chamber 160 to the interior (e.g., inside) of the pressure chamber 160 (e.g., via one or more of the first interface element 125 and/or the second interface element 124) in the administration device 100. In an implementation, the administration device 100 may include a sealed interface with the merging trigger to prevent, restrict, or regulate the flow of the pressurized fluid from the pressure chamber 160. This mode of operation may be available for administration routes including injection, oral, otic, intranasal, topical &c.

The activation system 150 may include the dispensing element 154, such as a needle for injection. Such a dispensing element 154 may be disposed between a distal passage 151 and a proximal passage 157. The distal passage 151 may communicate through a piston 159 to provide a path by which the dispensing element 154 may access the interior of a pressure chamber 160. The distal passage 151 may communicate through a distal activation support element 153, such as a clip, and the distal passage 151 may be open or closed responsive to the merged and/or armed state of the administration device 100. The proximal passage 157 may communicate through the proximal end 113 of the administration device 100 and provide a path by which the dispensing element 154 may access an ambient environment, including an injection site or other medical target site. The proximal passage 157 may communicate through a proximal activation support element 155, such as a clip or a fitment, and the proximal passage 157 may be open or closed responsive to the merged and/or armed state of the administration device 100. The activation system 150 may include a biasing element 152, such as a spring, and the biasing element 152 may provide a biasing force biasing the piston 159 in a distal direction to retain the piston 159 in an initial state until the activation system 150 is activated.

In an implementation, the biasing element 152 may be engaged with the compression element 126 to apply a biasing force to the compression element 126 to urge movement of the compression element 126. The administration device 100 may further include a retaining element configured to oppose the biasing force and retain the compression element 126 in a stationary state in an initial position. The retaining element may comprise the first interface element 125. The second interface element 124 may act on the retaining element to release the retaining element, causing the compression element 126 to move with respect to the storage assembly 130 (e.g., in response to the biasing force).

In another implementation, the biasing element 152 may be engaged with the carriage 121 to apply a biasing force to the carriage 121 to urge movement of the carriage 121. The administration device 100 may further include a retaining element configured to oppose the biasing force and retain the carriage 121 in a stationary state in an initial position. The retaining element may comprise the first interface element 125. The second interface element 124 may act on the retaining element to release the retaining element, causing the carriage 121 to move with respect to the storage assembly 130 (e.g., in response to the biasing force).

The administration device 100 may include a pressure chamber 160 configured to receive a pressurized fluid, and the storage assembly 130 may be disposed within the pressure chamber. The activation system may further comprise a pressurized fluid source 158, such as a canister, arranged such that action of the activation system 150 may actuate the pressurized fluid source 158 to provide a pressurized fluid into the pressure chamber 160, increasing the pressure within the pressure chamber 160. The increase in pressure may act on the reconstituted interior volume to urge the piston 159 proximally from an initial position to an administration position and allow fluid communication between the merged volume 139 and an ambient environment through the dispensing element 154. The activation system 150 may be configured to activate to dispense the beneficial agent by the elevated pressure in the pressure chamber (e.g., in the armed state). In this administration configuration, the administration device 100 may expel the reconstituted beneficial agent through the dispensing element 154.

In some arrangements, any storage assembly as described may comprise a rigid cartridge comprising a rigid walled hollow body comprising one or more plungers disposed therewithin to define boundaries of the compartments. In such arrangements, a compression element may be disposed for contact with one or more plungers to cause the merging of any interior volumes and mixing of any stored substances.

FIGS. 1e & 1g each illustrates the administration device 100 with the arming system and activation system more clearly shown. The arming system may be engageable with the activation system of the administration device 100. Such an arming system may be configured to transition the activation system between a safe state in which the activation system is configured to prevent activation of the administration device for dispensing the beneficial agent and an armed state in which the activation system is configured to allow activation of the administration device to dispensing the beneficial agent. The arming system may actuate to transition the activation system between the safe state and the armed state in conjunction with manipulation of the merging trigger 123 to cause merging/reconstitution, or the manipulation of the merging trigger 123 may be independent from the action of the arming system. The arming system may be configured to prevent transition to the armed state before the merging trigger 123 is manipulated to actuate the carriage 121 and merge the first compartment 136 and the second compartment 135 of the storage assembly 130, and the arming system may operate freely after the merging trigger 123 has been so manipulated. Thus, transitioning the device to the armed state may occur with merging, as a subsequent action after a timed delay without further user input, or may only occur after further user input, such as by twisting, rotating, and/or removing a cap. In implementations, the merging trigger is configured to be manipulable by a first manipulation (e.g., a rotation or a linear translation) to conduct the merging of the first interior volume and the second interior volume, and the merging trigger is manipulable by a second manipulation (e.g., a further same type or different type of manipulation) to cause the arming system to transition the activation system from the safe state to the armed state. In implementations, the activation system 150 is rendered manipulable by manipulation of the merging trigger 123.

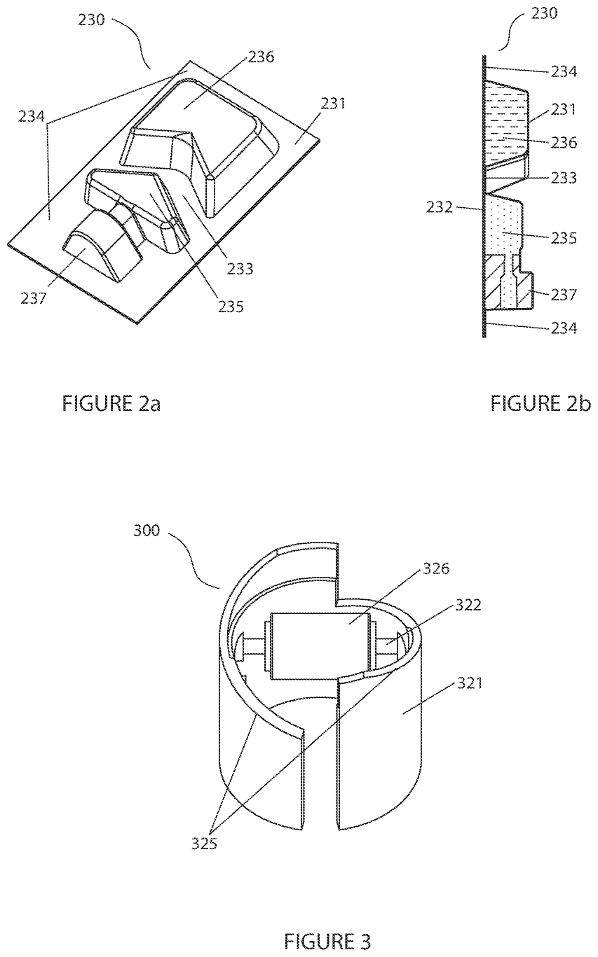

FIG. 2a & FIG. 2b illustrate respectively an over and a side view of a storage assembly 230 comprising a flexible package with a first compartment 236 including a first interior volume and a second compartment 235 including a second interior volume which are separated by a fluid isolation element 233 that can comprise a frangible seal. As shown, the first compartment 236 may include a concave geometry confronting the fluid isolation element 233. In other variations, the first compartment 236 may comprise a flat or convex geometry, and likewise for the second compartment 235. Modification of this geometry may serve to control the mixing of substances stored in the first compartment 236 and the second compartment 235. The storage assembly 230 may comprise a formed side 231, which may be composed of a formed polymer web composed of materials providing a high barrier to fluid ingress.

The storage assembly 230 may further comprise a lid side 232, which may be of the same or different material, such as an aluminum-backed polymer surface. A fluid isolation element 233, such as a frangible seal, may provide a barrier to fluid communication between the second storage volume of the second compartment 235 and the first storage volume of the first compartment 236. The second storage volume of the second compartment 235 may contain a second substance, such as a beneficial agent in dry form, and the first storage volume of the first compartment 236 may contain a first substance, such as a diluent like sterile water or a normal saline solution. Reconstitution may refer to the commencement of fluid communication between the compartments, to the formation of a mixed substance from a first substance and a second substance, or to the variation wherein a dry beneficial agent is mixed with a diluent. In some variations, both the first substance and the second substance may be in the same form (e.g., both may be wet or dry).

The storage assembly 230 can be modified to include more than two compartments and/or more than two stored substances in the pre-mixed state if desirable. A barrier 234 may be included in the storage assembly 230 to provide a durable seal and so may be sealed so as to not normally be breached when the fluid isolation element 233 is defeated during reconstitution or dosage administration. When the storage assembly 230 is compressed, the fluid isolation element 233 may be defeated, allowing fluid communication between the second compartment 235 and the first compartment 236. When merged, the interior volumes of the first compartment 236 and second compartment 235 may merge to form a merged volume. This volume may store a mixed substance, optionally for a period of hours to months, depending on formulation and need. A fitment 237 may be included and may be arranged to secure the storage assembly 230 within an administration device. Physical characteristics of the substance(s) contained within the first compartment 236 and the second compartment 235 may be controlled to promote rapid reconstitution, especially in the case of a dry beneficial agent to be reconstituted in a diluent and form a beneficial agent solution. The physical characteristics may include any of composition, particle size, particle shape, density, viscosity, or thermal characteristics such as specific heat or thermal stability.

FIG. 3 illustrates a carriage 300 comprising a carriage body 321 with an interface surface 325 comprising a cam follower. The carriage 300 may include a compression element 326 and a cross-member 322, which may support or include the compression element 326. The compression element 326 may include a roller, a rod, a panel, the cross-member, or a similar element configured to compress a compartment of a storage assembly when actuated by a merging trigger.

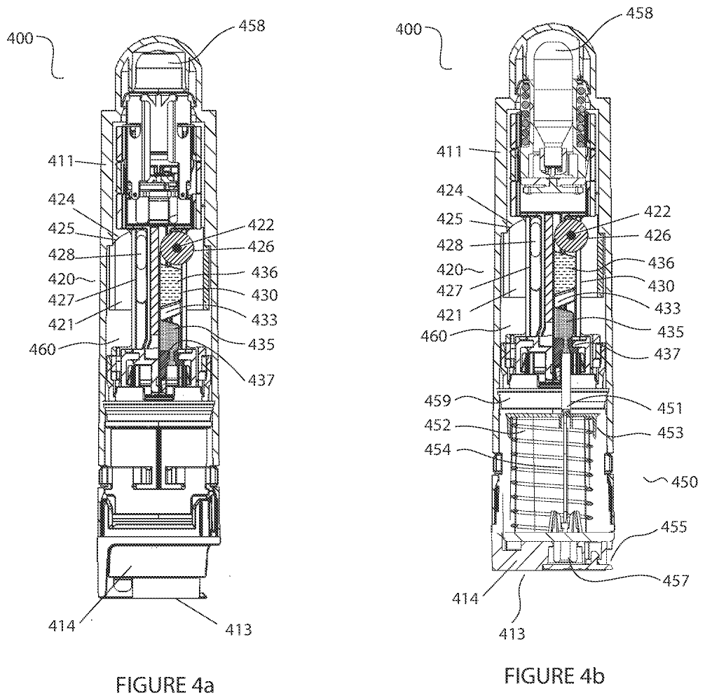

FIG. 4a & FIG. 4b illustrate an administration device 400 comprising an auto-injector and configured to reconstitute a beneficial agent stored in storage assembly 430 comprising a flexible primary drug container when the shell 411 is rotated relative to the proximal end 413 of the administration device 400. In this version of an administration device 400, the shell 411 may comprise a merging trigger operable as described and included as part of a merging mechanism 420 comprising the shell 411 and carriage 421. The shell 411 may include a first interface element 424 engageable with a second interface element 425 of a carriage 421 to move the carriage 421 relative to the storage assembly 430 in response to manipulation of the shell 411. The first interface element 424 may comprise a cam surface configured to rotate, and the second interface element 425 may comprise a cam follower configured to actuate linearly responsive to the rotation of the cam surface. The carriage 421 may include a compression element 426 and a cross-member 422, which may support or include the compression element 426. The compression element 426 may include a roller, a rod, a panel, the cross-member, or a similar element configured to compress a compartment of a storage assembly when actuated by a merging trigger.

The storage assembly 430 may include a fluid isolation element 433, such as a frangible seal, which may provide a barrier to fluid communication between the second storage volume of the second compartment 435 and the first storage volume of the first compartment 436. The second storage volume of the second compartment 435 may contain a second substance, such as an active ingredient of a beneficial agent in dry form, and the first storage volume of the first compartment 436 may contain a first substance, such as a diluent like sterile water or a normal saline solution. Reconstitution may refer to the commencement of fluid communication between the compartments, to the formation of a mixed substance from a first substance and a second substance, or to the variation wherein a dry beneficial agent is mixed with a diluent. In some variations, both the first substance and the second substance may be in the same form (e.g., both may be wet or dry). The storage assembly 430 can be modified to include more than two compartments and/or more than two stored substances in the pre-mixed state if desirable.

When a compartment of the storage assembly 430 is compressed, the fluid isolation element 433 may be defeated, allowing fluid communication between the second compartment 435 and the first compartment 436. When merged, the interior volumes of the first compartment 436 and second compartment 435 may merge to form a merged volume. This volume may store a mixed substance for a period ranging from minutes to hours to months, depending on the physical characteristics of the beneficial agent and the device application requirements. A fitment 437 may be included and may be arranged to secure the storage assembly 430 within the administration device 400. Physical characteristics of the substance(s) contained within the first compartment 436 and the second compartment 435 may be controlled to promote rapid mixing, especially in the case of a dry beneficial agent to be mixed in a diluent to form a beneficial agent solution. The physical characteristics may include any of composition, particle size, particle shape, density, viscosity, or thermal characteristics such as specific heat or thermal stability.

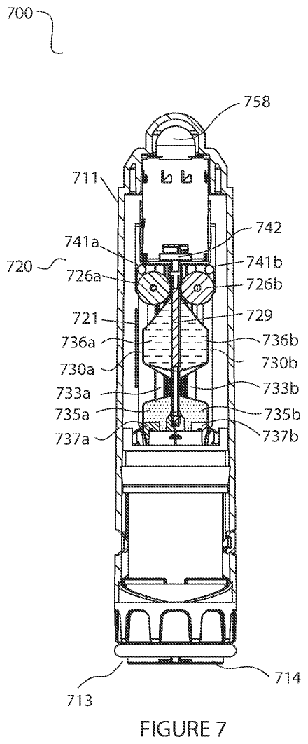

A cap 414 may be included in the administration device 400 and disposed at the proximal end 413 to cover a dispensing element 454, such as a needle. The shell 411 may be configured to rotate relative to the cap 414, engaging a first interface element 424 and a second interface element 425 as described to move the carriage 421 in the proximal direction. Movement of the carriage 421 relative to the storage assembly 430 may move the compression element 426 such that it will compress a compartment of the storage assembly 430 to defeat the fluid isolation element 433. With the defeat of the fluid isolation element 433, the interior volumes of the compartments may merge to form a merged volume. Substance(s) stored in the interior volumes may be reconstituted in the merged volume. A secondary follower 428 may be disposed within a guide slot 427 and configured to guide the linear actuation of the carriage 421. The secondary follower 428 and guide slot 427 may optionally be omitted, as in a dual-chamber administration device, such as that illustrated in FIG. 7, comprising a plurality of primary drug containers.

Manipulation of an arming trigger may manipulate an arming system engageable with an activation system 450 of the administration device 400. Manipulation of the merging trigger to activate the merging mechanism 420 may optionally be independent of the action of the arming system, although the merging trigger may comprise the arming trigger in some variations. The arming system may be configured to transition the activation system 450 between a safe state in which the activation system 450 is configured to prevent activation of the administration device 400 for dispensing the beneficial agent and an armed state in which the activation system 450 is configured to allow activation of the administration device 400 for dispensing the beneficial agent. The activation system 450 may include a pressurized fluid source 458, such as a canister containing a pressurized gas or liquid. In transitioning the activation system from the safe state to the armed state, the arming system may actuate the pressurized fluid source 458 to a position confronting a fluid source activator such as a rupturing pin. The configuration of the armed position may be such that the administration device 400 will activate and administer the beneficial agent to a target site when the proximal end 413 is pushed against the target site. The merging trigger may comprise an external portion that is external to the pressure chamber 460 and an internal portion disposed within the pressure chamber 460, the internal portion further comprising the second interface element. This mode of operation may be available for administration routes including injection, oral, otic, intranasal, topical &c.

The activation system 450 may include a dispensing element 454, such as an needle for injection. Such a dispensing element 454 may be disposed between a distal passage 451 and a proximal passage 457. The distal passage 451 may communicate through a piston 459 to provide a path by which the dispensing element 454 may access the interior of a pressure chamber 460. The distal passage 451 may communicate through a distal activation support element 453, such as a clip, and the distal passage 451 may be open or closed, responsive to the merged and/or armed state of the administration device 400. The proximal passage 457 may communicate through the proximal end 413 of the administration device 400 and provide a path by which the dispensing element 454 may access an ambient environment, including an injection site or other medical target site. The proximal passage 457 may communicate through a proximal activation support element 455, such as a clip or a fitment, and the proximal passage 457 may be open or closed responsive to the merged and/or armed state of the administration device 400. The activation system 450 may include a biasing element 452, such as a spring, and the biasing element 452 may provide a biasing force biasing the piston 459 in a distal direction to retain it in an initial state until the activation system 450 is activated.

The administration device 400 may include a pressure chamber 460 configured to receive a pressurized fluid, and the storage assembly 430 may be disposed within the pressure chamber. The activation system may further comprise a pressurized fluid source 458, such as a canister, arranged such that action of the activation system 450 may actuate the pressurized fluid source 458 to provide a pressurized fluid into the pressure chamber 460, increasing the pressure within the pressure chamber 460. The increase in pressure may act on the reconstituted interior volume to urge the piston 459 proximally from an initial position to an administration position and allow fluid communication between the merged volume and an ambient environment through the dispensing element 454. In this administration configuration, the administration device 400 may expel the reconstituted beneficial agent through the dispensing element 454.

The merging mechanism 420 may trigger an arming system engageable with an activation system 450 of the administration device 400. The arming system may be configured to transition the activation system 450 between a safe state in which the activation system 450 is configured to prevent activation of the administration device 400 for dispensing the beneficial agent and an armed state in which the activation system 450 is configured to allow activation of the administration device 400 for dispensing the beneficial agent. Alternatively, the merging mechanism 420 may be configured such that the manipulation of the shell 411 comprising the merging trigger is independent from the action of the arming system and/or the administration device 400 cannot be armed prior to reconstitution. For example, the administration device 400 may be arranged such that rotation of the cap 414 and/or proximal end 413 relative to the shell 411 actuates the carriage 421 to cause reconstitution, then in a separate action, the cap 414 engages the arming system to transition the activation system to the armed state when removed.

The administration device 400 may include a pressure chamber 460 configured to receive a pressurized fluid, and the storage assembly 430 may be disposed within the pressure chamber. The activation system may further comprise a pressurized fluid source 458, such as a canister, arranged such that action of the activation system 450 may actuate the pressurized fluid source 458 to provide a pressurized fluid into the pressure chamber 460, increasing the pressure within the pressure chamber 460. The increase in pressure may act on the reconstituted interior volume to expel the beneficial agent solution through the dispensing element 454.

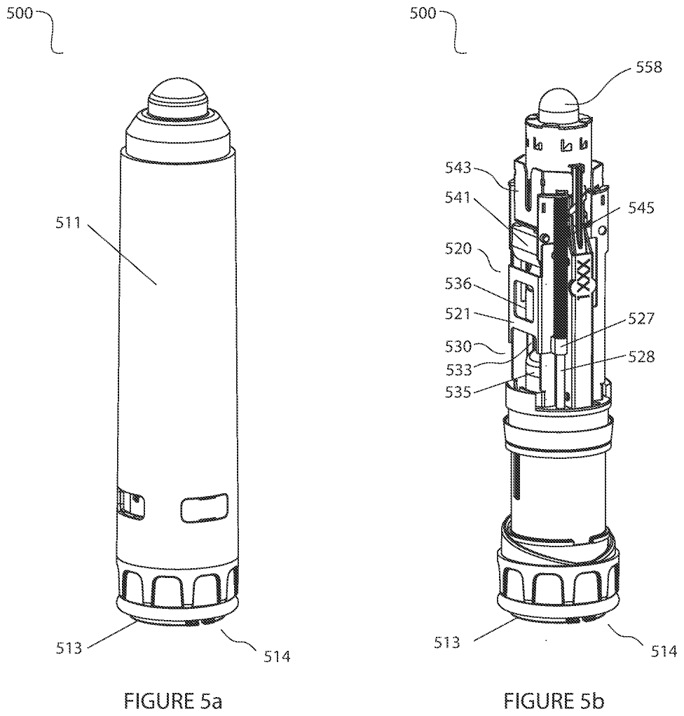

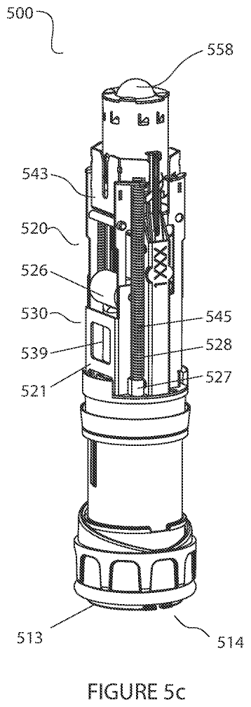

FIGS. 5a-c illustrate an administration device 500 comprising an auto-injector. FIG. 5a shows an external view of the administration device 500, including a shell 511 and a proximal end 513. FIG. 5b shows an internal view of the administration device 500 in the pre-reconstitution state, while FIG. 5c shows the post-reconstitution state. The administration device 500 may include a storage assembly 530 and a merging mechanism 520 disposed within the shell 511. The storage assembly 530 may comprise a flexible package with a first compartment 536 comprising a first interior volume and a second compartment 535 comprising a second interior volume.

In the pre-merged state of FIG. 5b, a fluid isolation element 533, such as a frangible seal, may provide a barrier to fluid communication between the respective interior volumes of second compartment 535 and the first compartment 536. This may serve to prevent mixing/reconstitution of a first substance in the first interior volume with a second substance in the second interior volume. Alternative versions of the administration device 500 may include more than two compartments in the pre-merged state of the storage assembly (e.g., two proximal compartments, each storing a separate dry substance and a distal compartment storing sufficient diluent to reconstitute both stored substances). The storage assembly may include a durable barrier providing a permanent seal configured to retain any stored or mixed substance against the ambient environment during the normal operation of the administration device 500. The storage assembly 530 may include a fitment configured to seat the storage assembly 530 within the administration device 500 at a well-defined and predictable location. When in fluid communication in the merged state, as shown in FIG. 5c, the first compartment 536 and second compartment 535 may combine to form a merged compartment 539 comprising a merged interior volume that may contain a reconstituted beneficial agent.

The merging mechanism 520 may be disposed within the shell 511 of the administration device 500 and may include a compression element 526, a strap 541, which may be configured to loop around the compression element 526, and a retaining element which may maintain the position of the strap 541. The merging mechanism 520 may be disposed partially within a plate 543 along with a pressurized fluid source 558, such as a canister. The merging mechanism 520 may include a carriage 521, a linear bearing 527, a carriage guide rod 528, and a biasing element 545, such as a spring. The biasing element 545 may be engaged with the carriage 521 to apply a biasing force to the carriage 521 to urge its movement. The retaining element may be a pin, and, along with the strap 541, may serve to oppose the biasing force to retain the carriage stationary in its initial position. The retaining element may comprise a first interface element. The merging mechanism 520 may function independently of an arming mechanism.

A cap 514 may be included in the administration device 500 and disposed at the proximal end 513 to cover a dispensing element such as a needle. The shell 511 may be configured to rotate relative to the cap 514, engaging a first interface element and a second interface element as described to move the carriage in the proximal direction. Movement of the carriage 521 relative to the storage assembly 530 may move the compression element 526 such that it will compress a compartment of the storage assembly 430 to defeat the fluid isolation element 433. With the fluid isolation element 433 being defeated, the interior volumes of the compartments may merge to form a merged volume. Substance(s) stored in the interior volumes may be reconstituted in the merged volume. A secondary follower 428 may be disposed within a guide slot 427 and configured to guide the linear actuation of the carriage 421.

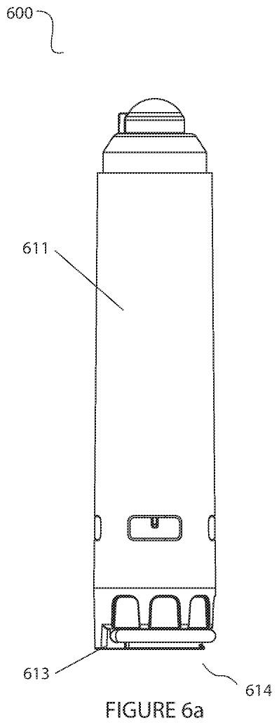

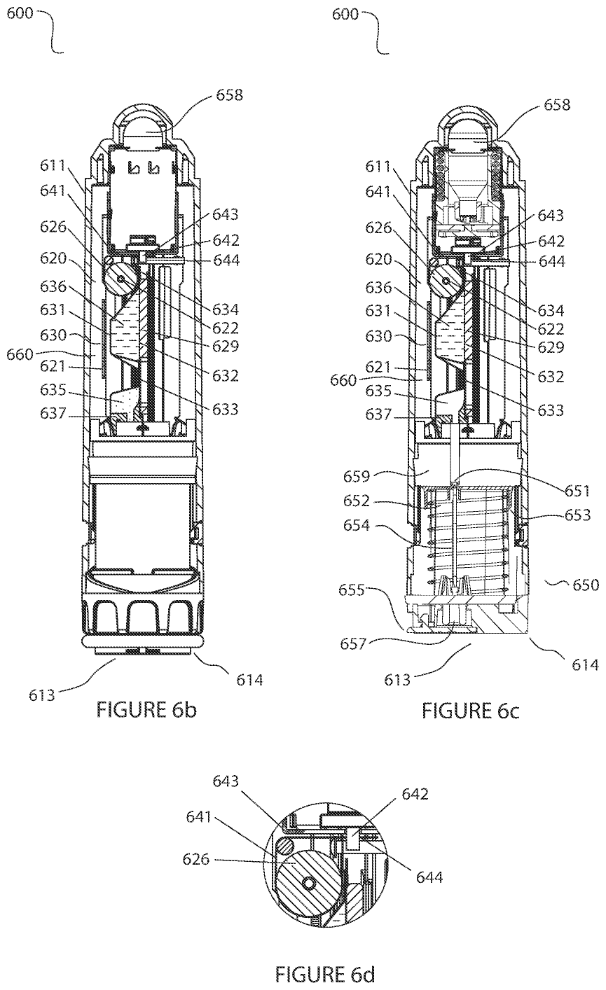

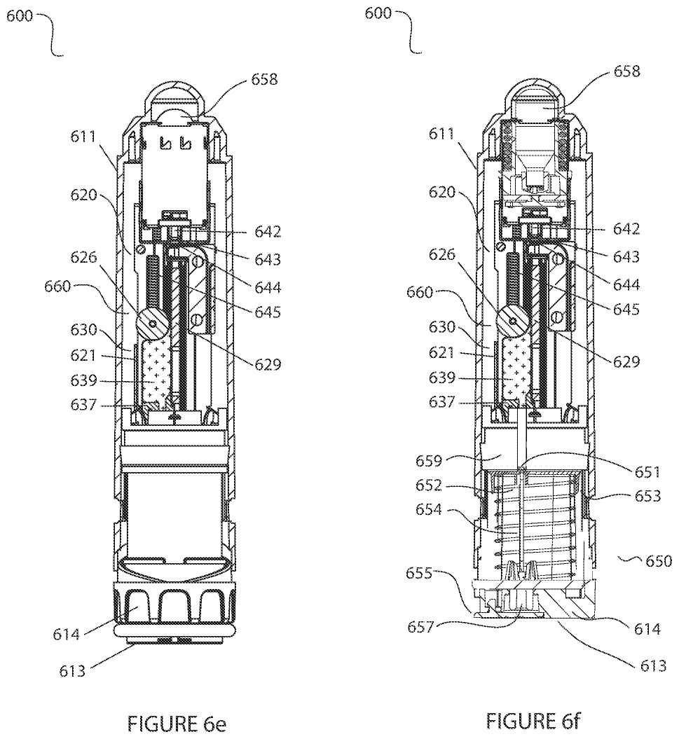

FIG. 6a illustrates a general exterior view of an administration device 600 comprising an auto-injector with a proximal end 613 and a shell 611 comprising the housing for the internal mechanisms of the device.

FIG. 6b & FIG. 6c illustrate an administration device 600 with a proximal end 613 in the initial state prior to triggering a merging trigger. In this initial state, the storage assembly 630 may comprise a first compartment 636 and a second compartment 635, and each compartment may include an interior volume that can store a substance prior to merging and/or mixing. The first compartment 636 and second compartment 635 may be separated by a fluid isolation element 633, such as a frangible seal, the storage assembly 630 may include a durable barrier 634 surrounding the compartments and sealing their interior volumes throughout the normal operation of the administration device 600. The storage assembly 630 may possess a formed side 631 and flat side 632 as described. The administration device 600 can include a merging mechanism 620, which may be disposed within the shell 611 and which may include a backing member 629. The storage assembly 630 may also include a fitment 637 configured to hold the storage assembly 630 in place within the administration device 600.

The merging mechanism 620 may include a carriage 62, a strap 641, and a compression element 626, such as a roller, panel, or cross-member. In the initial state, the merging mechanism may be disposed at a location distal to the storage assembly 630. The retaining element 642 may be arranged to secure the strap 641 and the compression element 626 in the initial position against a biasing force provided by a biasing element. The biasing element may be a spring held in tension or compression, and may be arranged to bias the carriage 621. The bias may be towards the proximal end 613 of the administration device 600. The retaining element 642 may be disposed within a hole 644 in the strap 641 and/or a plate 643 when in the initial position, and this may serve to prevent release of the strap 641, which would allow the carriage 621 to move. The carriage 621 may include a compression element 626 and a cross-member 622, which may support or include the compression element 626. The compression element 626 may include a roller, a rod, a panel, the cross-member, or a similar element configured to compress a compartment of a storage assembly when actuated by a merging trigger.

The retaining element 642 may comprise a first interface element engageable with a second interface element of a merging trigger, and manipulation of the merging trigger may engage the second interface element with the first interface element to release the retaining element 642. Either of the first interface element or the second interface element may comprise a cam surface, and the other of the first interface element or the second interface element may comprise a cam follower The merging mechanism 620 may be triggered by manipulation of a merging trigger, which may comprise the cap 614. The cap 614 may be disposed at a proximal end 613 of the administration device 600 and may be configured for manipulation such as twisting or rotational manipulation. The retaining element 642 may include a first interface element, and the merging trigger may comprise a second interface element engageable with the first interface element. Manipulation of the merging trigger may manipulate the second interface element against the first interface element to release the retaining element 642 and allow for movement of the carriage 621 from the initial position to a second position wherein the compression element 626 compresses a compartment of the storage assembly 630. Compression may defeat the fluid isolation element 633 of the storage assembly 630, causing the storage assembly 630 to transition to a merged state in which the interior volumes of the compartments are in fluid communication.

Manipulation of an arming trigger may manipulate an arming system engageable with an activation system 650 of the administration device 600. Manipulation of the merging trigger may optionally be independent of the action of the arming system, although the merging trigger may comprise the arming trigger in some variations. The arming system may be configured to transition the activation system 650 between a safe state in which the activation system 650 is configured to prevent activation of the administration device 600 for dispensing the beneficial agent and an armed state in which the activation system 650 is configured to allow activation of the administration device 600 for dispensing the beneficial agent. The activation system 650 may include a pressurized fluid source 658, such as a canister containing a pressurized gas or liquid. In transitioning the activation system from the safe state to the armed state, the arming system may actuate the pressurized fluid source 658 to a position confronting a fluid source activator such as a rupturing pin. The configuration of the armed position may be such that the administration device 600 will activate and administer the beneficial agent to a target site when the proximal end 613 is pushed against the target site. The merging trigger may comprise an external portion that is external to the pressure chamber 660 and an internal portion disposed within the pressure chamber 660, the internal portion further comprising the second interface element. This mode of operation may be available for administration routes including injection, oral, otic, intranasal, topical &c.

The activation system 650 may include a dispensing element 654, such as a needle for injection. Such a dispensing element 654 may be disposed between a distal passage 651 and a proximal passage 657. The distal passage 651 may communicate through a piston 659 to provide a path by which the dispensing element 654 may access the interior of a pressure chamber 660. The distal passage 651 may communicate through a distal activation support element 653, such as a clip, and the distal passage 651 may be open or closed, responsive to the merged and/or armed state of the administration device 600. The proximal passage 657 may communicate through the proximal end 613 of the administration device 600 and provide a path by which the dispensing element 654 may access an ambient environment, including an injection site or other medical target site. The proximal passage 657 may communicate through a proximal activation support element 655, such as a clip or a fitment, and the proximal passage 657 may be open or closed responsive to the merged and/or armed state of the administration device 600. The activation system 650 may include a biasing element 652, such as a spring, and the biasing element 652 may provide a biasing force biasing the piston 659 in a distal direction to retain it in an initial state until the activation system 650 is activated.

The administration device 600 may include a pressure chamber 660 configured to receive a pressurized fluid, and the storage assembly 630 may be disposed within the pressure chamber. The activation system may further comprise a pressurized fluid source 658, such as a canister, arranged such that action of the activation system 650 may actuate the pressurized fluid source 658 to provide a pressurized fluid into the pressure chamber 660, increasing the pressure within the pressure chamber 660. The increase in pressure may act on the reconstituted interior volume to urge the piston 659 proximally from an initial position to an administration position and allow fluid communication between the merged volume 639 and an ambient environment through the dispensing element 654. In this administration configuration, the administration device 600 may expel the reconstituted beneficial agent through the dispensing element 654.

In some arrangements, any storage assembly as described may comprise a rigid cartridge comprising a rigid walled hollow body comprising one or more plungers disposed therewithin to define boundaries of the compartments. In such arrangements, a compression element may be disposed for contact with one or more plungers to cause the merging of any interior volumes and mixing of any stored substances.

FIG. 6d shows a close-in view of a merging mechanism 620 comprising a compression element 626, such as a panel, roller, or cross-member, and a retaining element 642, such as a retaining pin disposed within a hole 644 in a strap 641 and/or a plate 643. The strap 641 may loop around the compression element 626 to hold it in the initial position against a biasing force provided by a biasing element. The retaining element 642 may serve, in turn, to retain the strap 641 against the biasing force of the biasing element transmitted into the strap 641 by the compression element 626. Manipulation of a merging trigger may serve to release the compression element 626 to move to the position illustrated in FIGS. 6e & 6f, wherein the compression element 626 applies a force to a compartment of a storage assembly 630.

FIG. 6e & FIG. 6f depict a side-on, cross-sectional view of the administration device 600 after the interior volumes of the first compartment 636 and the second compartment 635 have been merged such that they are in fluid communication. In such a merged state wherein the fluid isolation element is defeated, the merged interior volumes may comprise a merged volume 639 comprising a mixed substance formed by mixing the substances which were separated in the initial state. The storage assembly 630 may be disposed against the backing member 629 and may include a fitment 637 configured to maintain the storage assembly 630 in place within the administration device 600. The proximal end 613 of the administration device 600 may comprise a cap 614, which may be rotatable and/or removable.

Tension is released in the biasing element 645 by moving the retaining element 642 from a hole 644 present in the plate 643 and strap (not shown in this figure) transitioning the pin from a first state to a second state, permits translation of the carriage 621 and the compression element 626 in a proximal direction. The carriage 621 moves along a carriage guide rod (not shown in this figure) until the linear bearing (not shown in this figure) associated with the biasing element 645 comes to rest. The compression mechanism may comprise a compression element 626, such as a roller, a rod, a plate, or a similar element to squish a compartment of the storage assembly 630.

FIG. 7 illustrates an administration device 700 comprising an auto-injector configured for dual-injection. The administration device 700 includes a shell 711, a first storage assembly 730a, and a second storage assembly 730b. The administration device 700 includes a merging mechanism 720 capable of operating on both storage assemblies 730a-b. The merging mechanism 720 may include a carriage 721, first compression element 726a, and a second compression element 726b. The compression elements 726a-b may be retained in an initial position by straps 741a and 741b, which may be retained by a retaining element 742 as described. Some variations may comprise more than one retaining element. A rigid backing 729 may provide a structure against which the lid sides of the storage assemblies 730a-b may be disposed. The first storage assembly 730a may comprise a first fitment 737a, and the second storage assembly 730b may comprise a second fitment 737b.

The merging mechanism 720 may be triggered by manipulation of a merging trigger, which may comprise the cap 714. The cap 714 may be disposed at a proximal end 713 of the administration device 700 and may be configured for manipulation such as twisting or rotational manipulation. The retaining element 742 may include a first interface element, and the merging trigger may comprise a second interface element engageable with the first interface element. Manipulation of the merging trigger may manipulate the second interface element against the first interface element to release the retaining element 742 and allow for movement of the carriage 721 from the initial position to a second position wherein the compression elements 726a-b each compress a compartment of the storage assemblies 730a-b. Compression may defeat the first isolation element 733a and a second isolation element 733b, causing the storage assemblies 730a-b to transition to a merged state in which the interior volumes of the first compartment 736a of the first storage assembly 730a and the first compartment 736b of the second storage assembly 730b are in fluid communication with the interior volumes of the second compartment 735a of the first storage assembly 730a and the second compartment 735b of the second storage assembly 730b, respectively.

Manipulation of the merging trigger may manipulate an arming system engageable with an activation system of the administration device 700. The arming system may be configured to transition the activation system between a safe state in which the activation system is configured to prevent activation of the administration device 700 for dispensing the beneficial agent and an armed state in which the activation system is configured to allow activation of the administration device 700 for dispensing the beneficial agent. The activation system may include a pressurized fluid source 758, such as a canister containing a pressurized gas or liquid. In transitioning the activation system from the safe state to the armed state, the arming system may actuate the pressurized fluid source 758 to a position confronting a fluid source activator such as a rupturing pin. The configuration of the armed position may be such that the administration device 700 will activate and administer the beneficial agent to a target site when the proximal end 713 is pushed against the target site. This mode of operation may be available for administration routes including injection, oral, otic, intranasal, topical &c.

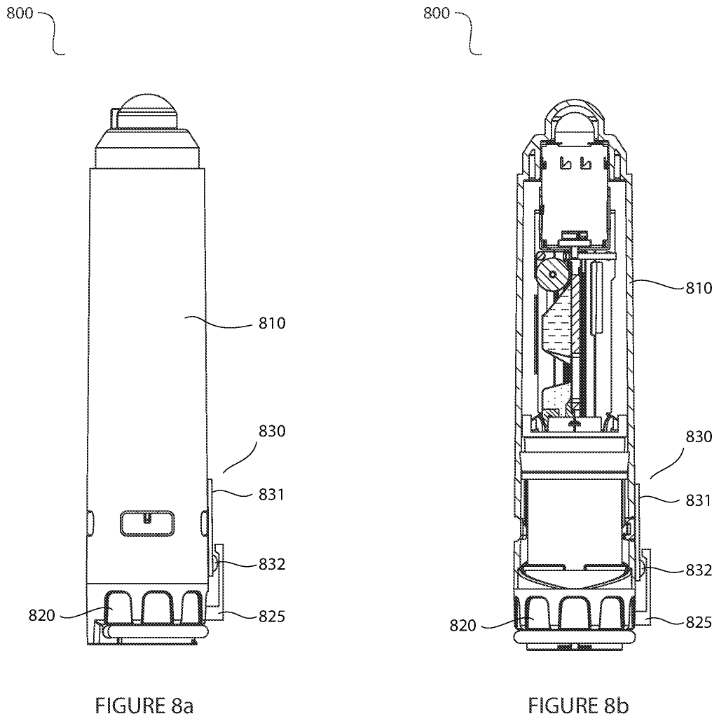

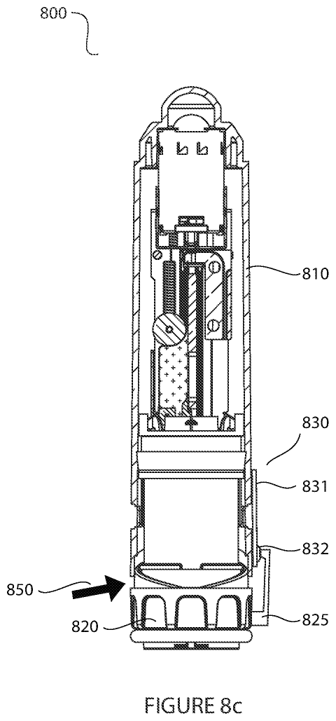

FIGS. 8a-c illustrate the activation of an elapsed time/temperature visualization that may be initiated by a response to manipulation of a cap 820 that depresses a button 832, resulting in a chemical, physical, enzymatic, or biological process that produces a visual output. This can measure a value such as: the time elapsed from activation, the time elapsed from activation within a defined temperature range, or a mean kinetic temperature. This information can be interpreted by observing a change in color, shape, pattern, or writing displayed on a reading window 831, allowing the user to determine the usability or non-usability of the mixed substance. Thus, the information related to the measurable value conveys if the reagent is fully reconstituted and safe, and ready to use or if the temporal window of administration has lapsed. In some configurations, the output may be in a tactile, visual, or visual and tactile form.

FIG. 8a shows an autoinjector 800, comprising a body 810, a cap 820, and a visual indicator 830. The cap 820 further comprises an actuating arm 825, and the visual indicator comprises a button 832 and a reading window 831. The autoinjector 800 is in a storage state prior to use. The cap 820 covers the administration end of the autoinjector 800 and is capable of two motions-the first motion moves the cap relative to the autoinjector body and the visual indicator while remaining attached to the body; the second motion is a removal of the cap from the body to expose the administration end where the needle is located, enabling deployment of the needle and injection. The reading window 831 serves to convey time/temperature information to the user.

FIG. 8b shows a cross-section of an autoinjector 800 comprising a body 810, a cap 820, and a visual indicator 830. The cap further comprises an actuating arm 825, and the visual indicator comprises a button 832 and a reading window 831. The autoinjector 800 is in a storage state. The actuating arm extends along the body, over the button angles towards the body such that moving the cap away from the body invariably causes the portion of the actuating arm protruding toward the body to press the actuating button.

FIG. 8c shows an autoinjector 800 comprising a body 810, a cap 820, and a visual indicator 830. The cap further comprises an actuating arm 825, and the visual indicator comprises a button 832 and a reading window 831. The autoinjector 800 is in the reconstitution state, and the visual indicator is in the actuated state. The cap was moved away from the body. The arrow 850 points to the gap that formed between the cap and the body as a result of the movement. The actuating arm is moved together with the cap and relative to the visual indicator, pressing the actuating button and causing the visual indicator to actuate at the same time as the reconstitution process was initiated. In some embodiments, the autoinjector may comprise an end cap (not shown) on the opposite end of the autoinjector body that serves as the element triggering reconstitution and actuating the visual indicator. In some embodiments, the visual indicator may be embedded in the body rather than being attached to the surface of the body. It may also be at least partially disposed inside the body, the reading strip visible through a transparent portion of the body. In other configurations, the actuating arm 825 may be moved by turning the cap.

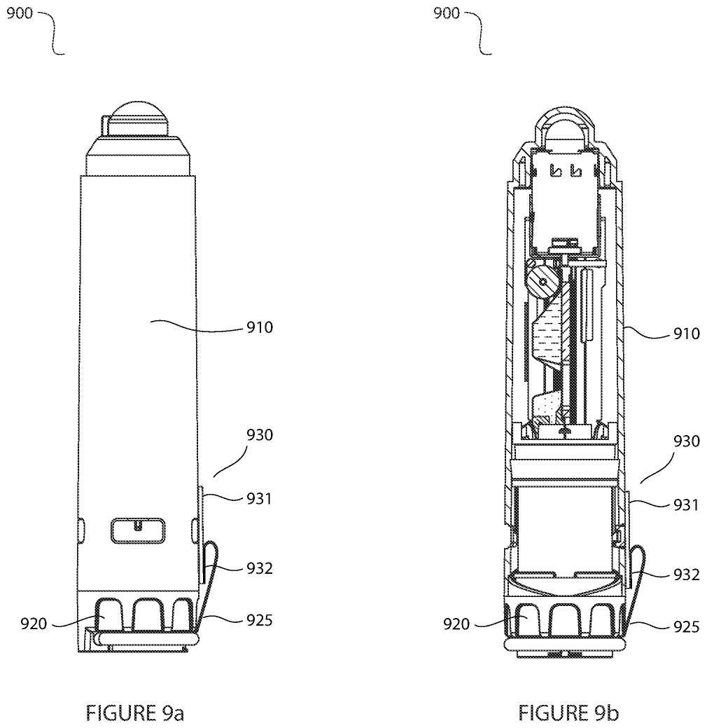

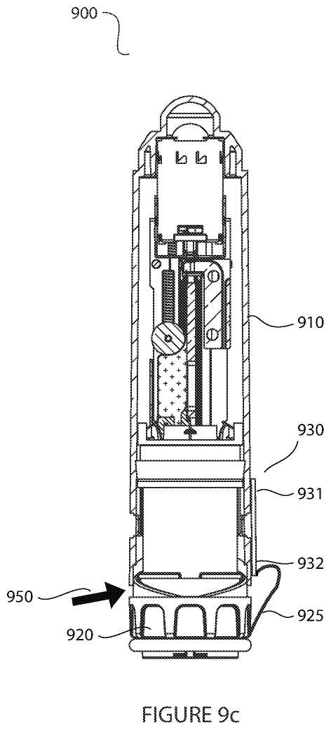

FIGS. 9a-c illustrate the activation of an elapsed time/temperature visualization that may be initiated by a response to manipulation of a cap 920 that tears a reactive strip 932 resulting in a chemical, physical, enzymatic, or biological process that produces a visual output. This can measure a value such as: the time elapsed from activation, the time elapsed from activation within a defined temperature range, or mean kinetic temperature. This information can be interpreted by observing a change in color, shape, pattern, or writing displayed on a reading window 931, allowing the user to determine the usability or non-usability of the mixed substance. Thus, the information related to the measurable value conveys if the reagent is fully reconstituted and safe, and ready to use or if the temporal window of administration has lapsed. In some configurations, the output may be in a tactile or visual and tactile form.

FIG. 9a shows an autoinjector 900, comprising a body 910, a cap 920, and a visual indicator 930. The cap further comprises an actuating strip 925, and the visual indicator comprises a reactive strip 932 and a reading window 931. The autoinjector 900 is in a storage state prior to use. The reactive strip is covered by the actuating strip, maintaining it in a non-reactive state.

FIG. 9b shows a cross-section of an autoinjector 900, comprising a body 910, a cap 920, and a visual indicator 930. The cap further comprises an actuating strip 925, and the visual indicator comprises a reactive strip 932 and a reading window 931. The autoinjector 900 is in a storage state prior to use. The reactive strip is covered by the actuating strip, maintaining it in a non-reactive state.

FIG. 9c shows a cross-section of an autoinjector 900, comprising a body 910, a cap 920, and a visual indicator 930. The cap further comprises an actuating strip 925, and the visual indicator comprises a reactive strip 932 and a reading window 931. The autoinjector is in a reconstitution state, and the visual indicator is in the actuated state. The cap was moved away from the body. The arrow 950 points to the gap that formed between the cap and the body as a result of the movement. The actuating strip moved together with the cap and relative to the visual indicator, peeling from and at least partially separating from the reactive strip. The reactive strip, when not covered by the actuating strip, actuates the visual indicator. This can occur by a reaction to light, O2, CO2, moisture, or other stimuli. The actuating strip may also trigger substances on the reactive strip that are kept separate in storage to come in contact. In other configurations, the actuating strip 925 may be moved by turning the cap.

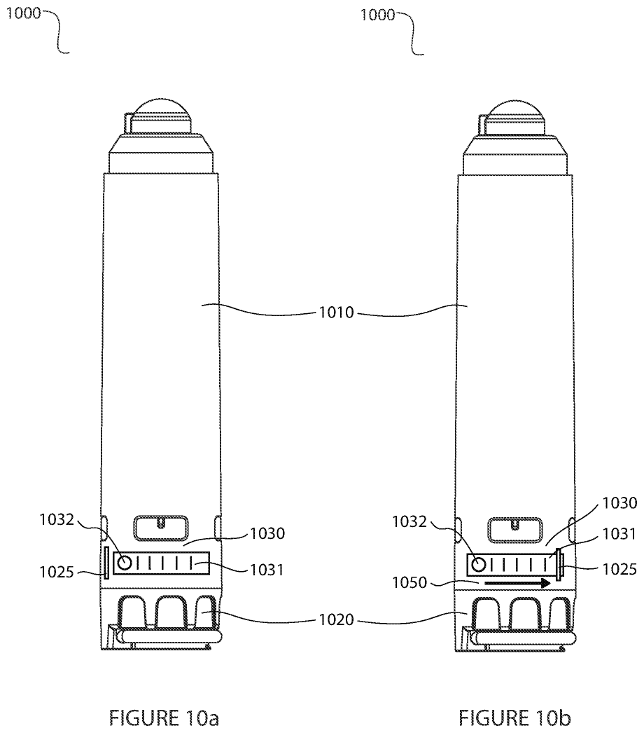

FIGS. 10a-b illustrate the activation of an elapsed time/temperature visualization that may be initiated by a response to manipulation of an actuating arm 1025 that moves across a button 1032, resulting in a chemical, physical, enzymatic, or biological process that produces a visual output. This can measure a value such as: the time elapsed from activation, the time elapsed from activation within a defined temperature range, or mean kinetic temperature. This information can be interpreted by observing a change in color, shape, pattern, or writing displayed on a reading window 1031, allowing the user to determine the usability or non-usability of the mixed substance. Thus, the information related to the measurable value conveys if the reagent is fully reconstituted and safe, and ready to use or if the temporal window of administration has lapsed. In some configurations, the output may be in a tactile or visual and tactile form.

FIG. 10a shows an autoinjector 1000 comprising a body 1010, a cap 1020, and a visual indicator 1030. The cap further comprises an actuating arm 1025, and the visual indicator comprises a button 1032 and a reading window 1031. The actuating arm extends along the body and along the visual indicator. The autoinjector is in a pre-reconstitution state and the visual indicator is in pre-actuation state.

FIG. 10b shows an autoinjector 1000 comprising a body 1010, a cap 1020, and a visual indicator 1030. The visual indicator comprises a button 1032 and a reading window 1031. Twisting of the cap relative to the body in the direction indicated by the arrow 1050 triggers reconstitution and actuation of the visual indicator 1030 by manipulating an actuating arm 1025. As the button 1032 protrudes away from the body 1010, the actuating arm must come in contact and deform the button 1032 to traverse it as it moves in the direction of the cap rotation.

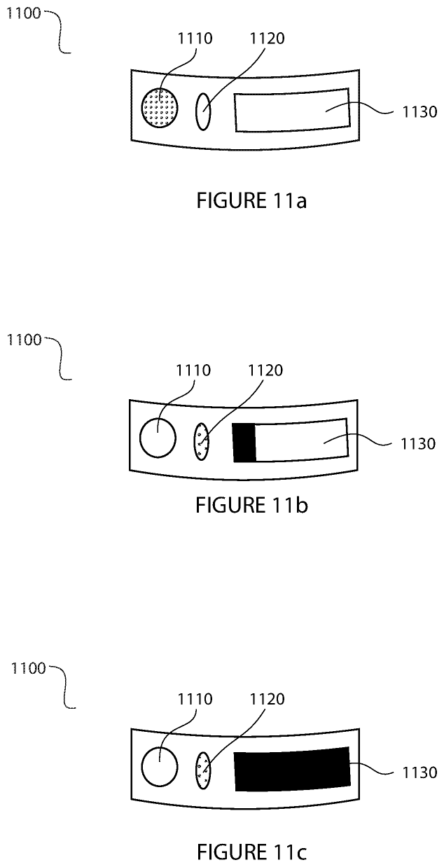

FIGS. 11a-c illustrate a pressure-actuated implementation of a visual indicator 1100, activated by pressure applied to a button-like activation element 1110 by an actuator that provides immediate feedback regarding the state (activated/inactivated) of the indicator. The purpose and function of the visual indicator 1100 are to provide the user with information regarding the readiness and safety of the beneficial agents within the administration device. In this embodiment, visualization can be seen to change over time rather than that of a binary indicator.

FIG. 11a shows a pressure-actuated implementation of a visual indicator 1100 in a pre-actuated state, similar to the visual indicators shown in FIGS. 8a-c and 10a-c. The visual indicator comprises a button-like activation element 1110, confirmation window 1120, and reading window 1130. The confirmation window 1120 serves to provide immediate feedback on the actuation status of the visual indicator to confirm successful actuation. A visual signal from the reading window 1130 may be slow, and in such embodiments, the confirmation window 1120 immediately gives the user confidence that the actuation has been successful.

FIG. 11b shows a pressure-actuated implementation of a visual indicator 1100 in an actuated state. The button-like activation element 1110 has been compressed, and the confirmation window has a different color to indicate actuation. A portion of the reading window 1130 has changed color to indicate that the reagent is in a safe and effective state for administration. The window may comprise vertical markings between the two horizontal ends, wherein if the first and the second color are present between the markings, it serves as a signal to the user to administer the drug.

FIG. 11c shows a pressure-actuated implementation of a visual indicator 1100 in an actuated state wherein the button-like activation element 1110 has been compressed, and the confirmation window displays a color different from what is visualized in the pre-actuated state. The entirety of the reading window 1130 has changed color to indicate that the period of safe and effective administration has passed.

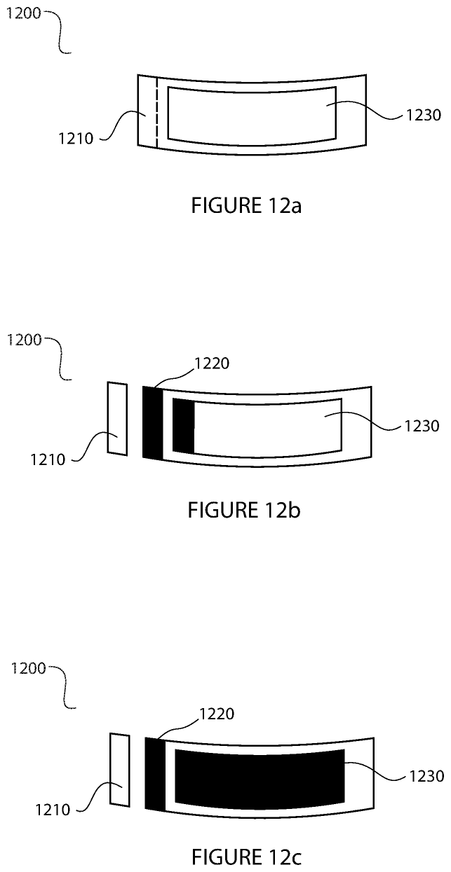

FIGS. 12a-c illustrate a pull-actuated implementation of a visual indicator 1200, activated by tearing of a tear-away activation element 1210, shown here as a tear-away strip, that provides immediate feedback regarding the state (activated/inactivated) of the indicator. The purpose and function of the visual indicator 1200 are to provide the user with information regarding the readiness and safety of the beneficial agents within the administration device. In this embodiment, visualization can be seen to change over time rather than that of a binary indicator.

FIG. 12a shows a pull-actuated implementation of a visual indicator 1200 in a pre-actuated state similar to the visual indicator shown in FIG. 9a-c. The visual indicator 1200 comprises a tear-away activation element 1210, confirmation strip 1220 (hidden under the tear-away strip), and reading window 1230. The confirmation strip 1220 serves to provide immediate feedback (e.g., generate a signal) on the actuation status of the visual indicator to confirm successful actuation (e.g., responsive to a manipulation of the merging trigger). A visual signal from the reading window 1230 may be slow, and in such embodiments, the confirmation strip 1220 immediately gives the user confidence that the actuation has been successful.

FIG. 12b shows the pull-actuated implementation of a visual indicator 1200 in an actuated state. The visual indicator 1200 comprises a tear-away activation element 1210, confirmation strip 1220, and reading window 1230. The tear-away strip has been separated from the rest of the visual indicator, exposing the confirmation strip. A portion of the reading window 1230 has changed color to indicate that the reagent is in a safe and effective state for administration. The window may comprise vertical markings between the two horizontal ends, wherein if the first and the second color are present between the markings, it serves as a signal to the user to administer the drug.

FIG. 12c shows the pull-actuated implementation of the visual indicator 1200 in an actuated state. The visual indicator 1200 comprises a tear-away activation element 1210, confirmation strip 1220, and reading window 1230. The tear-away strip has been separated from the rest of the visual indicator, exposing the confirmation strip. The entirety of the reading window 1230 has changed color to indicate the period of safe and effective administration has passed.

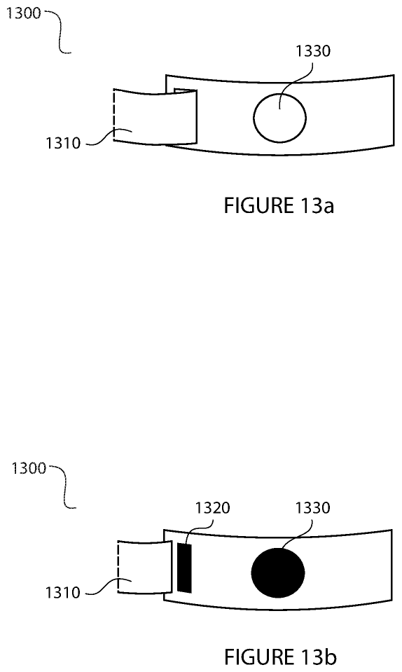

FIGS. 13a-b illustrate a visual indicator 1300, activated by tearing of a tear-away activation element 1310, that provides immediate feedback regarding the state (activated/inactivated) of the visual indicator 1300. The purpose and function of the visual indicator 1300 are to provide the user with information regarding the readiness and safety of the substances within the administration device. In this embodiment, visualization information is conveyed in a binary format (ready and safe/not fit for use).

FIG. 13a shows a pull-actuated implementation of a visual indicator 1300 in a pre-actuated state similar to the visual indicator shown in FIG. 9a-c. The visual indicator 1300 comprises a tear-away activation element 1310, confirmation strip 1320 (hidden under the tear-away strip), and reading window 1330. The confirmation strip 1320 serves to provide immediate feedback on the actuation status of the visual indicator to confirm successful actuation. A visual signal from the reading window 1330 may be slow, and in such embodiments, the confirmation strip 1320 immediately gives the user confidence that the actuation has been successful. In the illustrated implementation, the visual indicator 1300 is a binary indicator that indicates the useability or non-usability of a mixed substance comprising the beneficial agent.

FIG. 13b shows the pull-actuated implementation of the visual indicator 1300 in an actuated state. The visual indicator 1300 comprises a tear-away activation element 1310, confirmation strip 1320, and reading window 1330. The tear-away strip has been separated from the rest of the visual indicator, exposing the confirmation strip. As illustrated, the entirety of the reading window 1330 has changed color to indicate that the reagent is in a safe and effective state for administration.

Clause 1. An administration device for administering a beneficial agent, comprising: a storage assembly comprising a first compartment and a second compartment, the first compartment fluidly separated from the second compartment by a fluid isolation element that isolates a first interior volume of the first compartment from a second interior volume of the second compartment; a merging mechanism, comprising: a first interface element; a merging trigger comprising a second interface element, the second interface element being engageable with the first interface element to move relative to the storage assembly in response to manipulation of the merging trigger; and a compression element supportably engaged to the first interface element and moveable therewith, the compression element being disposed for contact of at least one of the first compartment or the second compartment in response to movement of the first interface element relative to the storage assembly to defeat the fluid isolation element and merge the first interior volume and the second interior volume; and an arming system engageable with an activation system of the administration device, the arming system configured to transition the activation system between a safe state in which the activation system is configured to prevent activation of the administration device for dispensing the beneficial agent and an armed state in which the activation system is configured to allow activation of the administration device for dispensing the beneficial agent; wherein the manipulation of the merging trigger is independent from action of the arming system.

Clause 2. The administration device of clause 1, the merging mechanism further comprising: a carriage comprising the first interface element, the second interface element being engageable with the first interface element to move the carriage relative to the storage assembly in response to manipulation of the merging trigger; wherein the compression element is supportably engaged by the carriage and moveable therewith, the compression element being disposed for contact of at least one of the first compartment or the second compartment in response to movement of the carriage relative to the storage assembly.

Clause 3. The administration device of clause 2, wherein one of the first interface element or the second interface element comprises a cam surface, the other of the first interface element or the second interface element comprises a cam follower, and wherein manipulation of the merging trigger results in relative movement of the cam surface and the cam follower to move the carriage relative to the storage assembly.

Clause 4. The administration device of clause 3, wherein the cam surface and the cam follower undergo the relative movement in response to rotational movement of the merging trigger.

Clause 5. The administration device of clause 4, wherein movement of the carriage relative to the storage assembly is linear movement.

Clause 6. The administration device of clause 5, wherein the cam surface and the cam follower comprise opposing inclined surfaces, relative rotation between the opposing inclined surfaces resulting in the linear movement of the carriage.

Clause 7. The administration device of clause 1, wherein the first interior volume comprises a first substance, the second interior volume comprises a second substance, and merging of the first interior volume and the second interior volume creates a reconstituted interior volume of the storage assembly contains a beneficial agent solution comprising the first substance and the second substance.

Clause 8. The administration device of clause 7, wherein the first substance comprises a diluent and the second substance comprises an active ingredient of the beneficial agent.

Clause 9. The administration device of clause 7, wherein the activation system, when in the armed state, is configured to allow activation of the administration device to expel the beneficial agent solution from the reconstituted interior volume through a dispensing element of the administration device.

Clause 10. The administration device of clause 7, further comprising: a pressure chamber configured to receive a pressurized fluid, wherein the storage assembly is disposed within the pressure chamber; and wherein the activation system further comprises a pressurized fluid source arranged such that action of the activation system actuates the pressurized fluid source to provide a pressurized fluid to the pressure chamber to increase a pressure within the pressure chamber; wherein the increase in pressure acts on the reconstituted interior volume to expel the beneficial agent solution through a dispensing element of the administration device.

Clause 11. The administration device of clause 10, wherein the merging trigger comprises an external portion that is external to the pressure chamber and an internal portion disposed within the pressure chamber, the internal portion further comprising the second interface element.

Clause 12. The administration device of clause 10, further comprising: a sealed interface with the merging trigger to prevent or regulate flow of the pressurized fluid from the pressure chamber.

Clause 13. The administration device of clause 1, further comprising: an arming trigger configured to, in response to manipulation of the arming trigger, initiate the transition of the activation system between the safe state and the armed state.

Clause 14. The administration device of clause 13, wherein the arming system is not acted upon by manipulation of the merging mechanism.

Clause 15. The administration device of clause 13, wherein actuation of the arming trigger is substantially prevented prior to manipulation of the merging trigger and is substantially not prevented after manipulation of the merging trigger.

Clause 16. The administration device of clause 1, wherein the merging trigger is manipulable by a first manipulation to conduct the merging of the first interior volume and the second interior volume, and the merging trigger is manipulable by a second manipulation to cause the arming system to transition the activation system from the safe state to the armed state.

Clause 17. The administration device of clause 1, wherein the compression element is configured for compression of at least one of the first compartment or the second compartment to defeat the fluid isolation element.

Clause 18. The administration device of clause 17, wherein the compression element comprises at least one of a cross-member, a rod, a panel, or a roller, configured to increase pressure in at least one of the first or the second compartment to defeat the fluid isolation element.

Clause 19. The administration device of clause 1, wherein the storage assembly comprises a flexible package, the fluid isolation element comprising a frangible seal disposed between the first interior volume and the second interior volume.

Clause 20. The administration device of clause 19, wherein the storage assembly includes a first wall and a second wall sealed against the first wall to define the first interior volume, the second interior volume, and the frangible seal.

Clause 21. The administration device of clause 19, wherein the storage assembly further comprises a port configured to communicate the beneficial agent from the storage assembly to a dispensing element of the administration device upon activation of the activation system.

Clause 22. The administration device of clause 19, wherein the storage assembly further comprises a third interior volume separate from the first interior volume and the second interior volume prior by a second fluid isolation element, the third interior volume containing a constituent of the beneficial agent different from contents of the first interior volume and the second interior volume, the contact of the compression element further configured to defeat the second fluid isolation element and merge the third interior volume with the first interior volume and the second interior volume.

Clause 23. The administration device of clause 1, wherein the merging trigger includes any one or more of a cap, knob, lever, or button.

Clause 24. The administration device of clause 1, wherein the merging trigger comprises at least a portion of a shell of the administration device.

Clause 25. The administration device of clause 1, further comprising an indicator that is configured to generate a signal responsive to manipulation of the merging trigger.

Clause 26. The administration device of clause 25, wherein the signal comprises at least one of a visual indication or a tactile indication.

Clause 27. The administration device of clause 26, wherein the signal conveys information related to a measurable value.

Clause 28. The administration device of clause 27, wherein the measurable value includes any one or more of a first elapsed time from merging, a second elapsed time from merging within a defined temperature range, or a mean kinetic temperature.

Clause 29. The administration device of clause 28, wherein the signal is generated by any one or more of a chemical process, a physical process, an enzymatic process, or a biological process.

Clause 30. The administration device of clause 25, wherein the signal includes a binary indication of usability or non-usability of a mixed substance comprising the beneficial agent.

Clause 31. The administration device of clause 25, wherein the activation system is rendered manipulable by the manipulation of the merging trigger.

Clause 32. The administration device of clause 1, wherein the manipulation of the merging trigger includes rotation or linear motion of the merging trigger.

Clause 33. The administration device of clause 1, wherein the arming system is configured to transition the activation system between the safe state and the armed state based on a rotation or linear motion of a first portion of the administration device relative to a second portion of the administration device.

Clause 34. The administration device of clause 1, wherein the merging trigger is manipulable by rotation or linear motion to activate the activation system in the armed state.

Clause 35. The administration device of clause 1, wherein the activation system is manipulable by rotation or linear motion to activate the activation system.

Clause 36. An administration device for administering a beneficial agent, comprising: a storage assembly comprising a first compartment and a second compartment, the first compartment being separably maintained from the second compartment by a fluid isolation element that isolates a first interior volume of the first compartment from a second interior volume of the second compartment; a merging mechanism, comprising: a first interface element; a merging trigger comprising a second interface element, the second interface element being engageable with the first interface element to move the first interface element relative to the storage assembly in response to rotational manipulation of the merging trigger; and a compression element supportably engaged by the first interface element and moveable therewith, the compression element being disposed for contact of at least one of the first compartment or the second compartment in response to movement of the first interface element relative to the storage assembly to defeat the fluid isolation element and merge the first interior volume and the second interior volume.

Clause 37. The administration device of clause 36, the merging mechanism further comprising: a carriage comprising the first interface element, the second interface element being engageable with the first interface element to move the carriage relative to the storage assembly in response to manipulation of the merging trigger; wherein the compression element is supportably engaged by the carriage and moveable therewith, the compression element being disposed for contact of at least one of the first compartment or the second compartment in response to movement of the carriage relative to the storage assembly.

Clause 38. The administration device of clause 37, wherein one of the first interface element or the second interface element comprises a cam surface, the other of the first interface element or the second interface element comprises a cam follower, and wherein manipulation of the merging trigger results in relative movement of the cam surface and the cam follower to move the carriage relative to the storage assembly.

Clause 39. The administration device of clause 38, wherein the cam surface and the cam follower undergo the relative movement in response to rotational movement of the merging trigger.

Clause 40. The administration device of clause 39, wherein movement of the carriage relative to the storage assembly is linear movement.

Clause 41. The administration device of clause 40, wherein the cam surface and the cam follower comprise opposing inclined surfaces, relative rotation between the opposing inclined surfaces resulting in the linear movement of the carriage.

Clause 42. The administration device of clause 36, further comprising: a biasing element engaged with the compression element to apply a biasing force to the compression element to urge movement of the compression element; and a retaining element configured to oppose the biasing force to retain the compression element stationary in an initial position, the retaining element comprising the first interface element; wherein the second interface element acts on the retaining element to release the retaining element, causing the compression element to move with respect to the storage assembly in response to the biasing force.

Clause 43. The administration device of clause 37, further comprising: a biasing element engaged with the carriage to apply a biasing force to the carriage to urge movement of the carriage; and a retaining element configured to oppose the biasing force to retain the carriage stationary in an initial position, the retaining element comprising the first interface element; wherein the second interface element acts on the retaining element to release the retaining element, causing the carriage to move with respect to the storage assembly in response to the biasing force.

Clause 44. The administration device of clause 36, wherein the first interior volume comprise a first substance, the second interior volume comprises a second substance, and merging of the first interior volume and the second interior volume creates a reconstituted interior volume of the storage assembly containing a beneficial agent solution comprising the first substance and the second substance.

Clause 45. The administration device of clause 44, wherein the first substance comprises a first constituent of the beneficial agent and the second substance comprises a second constituent of the beneficial agent.

Clause 46. The administration device of clause 44, further comprising: an arming system engageable with an activation system of the administration device, the arming system configured to transition the activation system between a safe state in which the activation system is configured to prevent activation of the administration device for dispensing the beneficial agent and an armed state in which the activation system is configured to allow activation of the administration device for dispensing the beneficial agent.

Clause 47. The administration device of clause 46, wherein the activation system, when in the armed state, is configured for activating the administration device to expel the beneficial agent solution from the reconstituted interior volume through a dispensing element of the administration device.

Clause 48. The administration device of clause 46, wherein the arming system is configured to transition the activation system between the safe state and the armed state based on a rotation or linear motion of a first portion of the administration device relative to a second portion of the administration device.

Clause 49. The administration device of clause 46, wherein the merging trigger is manipulable by rotation or linear motion to activate the activation system in the armed state.

Clause 50. The administration device of clause 46, wherein the activation system is manipulable by rotation of linear motion to activate the activation system.

Clause 51. The administration device of clause 46, wherein the merging trigger is manipulable by a first manipulation to conduct the merging of the first interior volume and the second interior volume, and the merging trigger is manipulable by a second manipulation to cause the arming system to transition the activation system from the safe state to the armed state.

Clause 52. The administration device of clause 46, further comprising: a pressure chamber configured to receive a pressurized fluid, wherein the storage assembly is disposed within the pressure chamber; and wherein the activation system further comprises a pressurized fluid source arranged such that action of the activation system actuates the pressurized fluid source to provide a pressurized fluid to the pressure chamber to increase a pressure within the pressure chamber; wherein the increase in pressure acts on the reconstituted interior volume to dispense the beneficial agent solution through a dispensing element.

Clause 53. The administration device of clause 52, wherein the merging trigger comprises an external portion that is external to the pressure chamber and an internal portion disposed within the pressure chamber, the internal portion further comprising the second interface element.

Clause 54. The administration device of clause 52, further comprising: a sealed interface with the merging trigger to prevent or regulate flow of the pressurized fluid from the pressure chamber.

Clause 55. The administration device of clause 46, further comprising: an arming trigger configured to, in response to manipulation of the arming trigger, initiate the transition of the activation system between the safe state and the armed state.

Clause 56. The administration device of clause 55, wherein the arming trigger is not acted upon by manipulation of the merging trigger.

Clause 57. The administration device of clause 55, wherein the merging trigger comprises the arming trigger.

Clause 58. The administration device of clause 55, wherein actuation of the arming trigger is substantially prevented prior to manipulation of the merging trigger and is substantially not prevented after manipulation of the merging trigger.

Clause 59. The administration device of clause 36, wherein the compression element is configured for compression of at least one of the first compartment or the second compartment to defeat the fluid isolation element.

Clause 60. The administration device of clause 59, wherein the compression element comprises at least one of a cross-member, a panel, or a roller.

Clause 61. The administration device of clause 36, wherein the storage assembly comprises a flexible package, the fluid isolation element comprising a frangible seal disposed between the first interior volume and the second interior volume.

Clause 62. The administration device of clause 61, wherein the storage assembly includes a first wall and a second wall sealed against the first wall to define the first interior volume, the second interior volume, and the frangible seal.

Clause 63. The administration device of clause 61, wherein the storage assembly further comprises a port configured to communicate the beneficial agent from the storage assembly to a dispensing element of the administration device.

Clause 64. The administration device of clause 61, wherein the storage assembly further comprises a third interior volume separated from the first interior volume and the second interior volume by a second fluid isolation element, the third interior volume containing a constituent of the beneficial agent different from contents of the first interior volume and the second interior volume, the contact of the compression element further configured to defeat the second fluid isolation element and merge the third interior volume with the first interior volume and the second interior volume.

Clause 65. The administration device of clause 36, wherein the merging trigger includes any one or more of a cap, knob, lever, or button.

Clause 66. The administration device of clause 36, wherein the merging trigger comprises at least a portion of a shell of the administration device.

Clause 67. The administration device of clause 46, further comprising an indicator that is configured to generate a signal responsive to manipulation of the activation system.

Clause 68. The administration device of clause 67, wherein the signal comprises at least one of a visual indication or a tactile indication.

Clause 69. The administration device of clause 68, wherein the signal conveys information related to a measurable value.

Clause 70. The administration device of clause 69, wherein the measurable value includes any one or more of a first elapsed time from activation, a second elapsed time from activation within a defined temperature range, or a mean kinetic temperature.

Clause 71. The administration device of clause 70, wherein the signal is generated by any one or more of a chemical process, a physical process, an enzymatic process, or a biological process.

Clause 72. The administration device of clause 67, wherein the signal includes a binary indication of usability or non-usability of a mixed substance comprising the beneficial agent.

Clause 73. The administration device of clause 67, wherein the activation system is manipulable responsive to actuation of the merging trigger.

Clause 74. An administration device for administering a beneficial agent, comprising: a storage assembly comprising a first compartment and second compartment; a merging mechanism comprising: a compression element; a biasing element engaged with the compression element to apply a biasing force to the compression element to urge movement of the compression element; and a retaining element configured to oppose the biasing force to retain the compression element stationary in an initial position; wherein manipulation of a merging trigger releases the retaining element to transition the storage assembly from an initial state in which the first compartment and the second compartment are separate to a merged state in which the first compartment and the second compartment are in fluid communication in response to movement of the compression element relative to the storage assembly.

Clause 75. The administration device of clause 74, the merging mechanism further comprising: a carriage comprising the compression element, wherein the biasing element is engaged with the carriage to apply a biasing force to the carriage to urge movement of the carriage, and the retaining element is configured to oppose the biasing force to retain the carriage stationary in an initial position.

Clause 76. The administration device of clause 74, further comprising: an arming system engageable with an activation system and configured to, in response to manipulation of an arming trigger, transition the activation system between a safe state in which the activation system is configured to prevent activation of the administration device for dispensing the beneficial agent and an armed state in which the activation system is configured to allow the activation of the administration device for dispensing the beneficial agent.

Clause 77. The administration device of clause 76, wherein the merging trigger comprises the arming trigger.

Clause 78. The administration device of clause 77, wherein the arming trigger resists actuation when the administration device is in the initial state and is free to actuate when the administration device is in the merged state.

Clause 79. The administration device of clause 76, wherein the arming system is configured to transition the activation system between the safe state and the armed state based on a rotation or linear motion of a first portion of the administration device relative to a second portion of the administration device.

Clause 80. The administration device of clause 76, wherein the merging trigger is manipulable by rotation or linear motion to activate the activation system in the armed state.

Clause 81. The administration device of clause 76, wherein the activation system is manipulable by rotation of linear motion to activate the activation system.

Clause 82. The administration device of clause 76, wherein the merging trigger is manipulable by a first manipulation to conduct the merging of the first compartment and the second compartment, and the merging trigger is manipulable by a second manipulation to cause the arming system to transition the activation system from the safe state to the armed state.

Clause 83. The administration device of clause 74, wherein the merging trigger comprises first interface element and the merging mechanism comprises a second interface element, the manipulation of the merging trigger engaging the first interface element and the second interface element to release the retaining element.

Clause 84. The administration device of clause 83, wherein one of the first interface element or the second interface element comprises a cam surface, the other of the first interface element or the second interface element comprises a cam follower.

Clause 85. The administration device of clause 74, wherein the compression element comprises at least one of a cross-member, a panel, or a roller.

Clause 86. The administration device of clause 74, wherein the storage assembly comprises a frangible seal, the frangible seal disposed between the first compartment and the second compartment to fluidly isolate the first compartment and the second compartment in the initial state.

Clause 87. The administration device of clause 86, wherein the frangible seal is configured to separate to provide fluid communication between the first compartment and the second compartment responsive to contact of the compression element with at least one of the first compartment or the second compartment.

Clause 88. The administration device of clause 74, further comprising: a first substance stored in the first compartment in the initial state; and a second substance stored in the second compartment in the initial state; wherein the first substance and the second substance are mixable to form a mixed substance when the storage assembly is in the merged state.

Clause 89. The administration device of clause 88, wherein the first substance comprises a diluent and the second substance comprises a beneficial agent.

Clause 90. The administration device of clause 74, wherein the merging trigger comprises any one or more of a cap, knob, lever, or button.

Clause 91. The administration device of clause 74, wherein the merging trigger comprises at least a portion of a shell of the administration device.

Clause 92. The administration device of clause 74, wherein the manipulation of the merging trigger includes rotation or linear motion of the merging trigger.

Clause 93. An administration device for administering a beneficial agent, comprising: a pressure chamber; a storage assembly disposed within the pressure chamber, comprising: a first compartment; and a second compartment, the first compartment fluidly separated from the second compartment by a fluid isolation element that isolates a first interior volume of the first compartment from a second interior volume of the second compartment; a merging mechanism, comprising: a merging trigger; and a compression element being disposed for contact of at least one of the first compartment or the second compartment in response to movement of the merging trigger relative to the storage assembly to defeat the fluid isolation element and merge the first interior volume and the second interior volume; and an arming system engageable with an activation system of the administration device, the arming system configured to transition the activation system between a safe state in which the activation system is configured to prevent activation of the administration device for dispensing the beneficial agent and an armed state in which the activation system is configured to allow activation of the administration device for dispensing the beneficial agent; wherein the movement of the merging mechanism is independent from action of the arming system.

Clause 94. The administration device of clause 93, wherein the merging trigger is configured to transfer a motion from outside the pressure chamber to inside the pressure chamber.

Clause 95. The administration device of clause 93, wherein the activation system is configured to activate to dispense the beneficial agent by elevating pressure in the pressure chamber in the armed state.

Clause 96. The administration device of clause 93, the merging mechanism further comprising: a first interface element, wherein: the merging trigger comprises a second interface element, the second interface element being engageable with the first interface element to move relative to the storage assembly in response to manipulation of the merging trigger; the compression element is supportably engaged to the first interface element and moveable therewith; and the compression element being disposed for contact of the at least one of the first compartment or the second compartment in response to movement of the first interface element responsive to the movement of the merging trigger.

Clause 97. The administration device of clause 96, the merging mechanism further comprising: a carriage comprising the first interface element, the second interface element being engageable with the first interface element to move the carriage relative to the storage assembly in response to manipulation of the merging trigger; wherein the compression element is supportably engaged by the carriage and moveable therewith, the compression element being disposed for contact of at least one of the first compartment or the second compartment in response to movement of the carriage relative to the storage assembly.

Clause 98. The administration device of clause 97, wherein one of the first interface element or the second interface element comprises a cam surface, the other of the first interface element or the second interface element comprises a cam follower, and wherein manipulation of the merging trigger results in relative movement of the cam surface and the cam follower to move the carriage relative to the storage assembly.

Clause 99. The administration device of clause 98, wherein the cam surface and the cam follower undergo the relative movement in response to rotational movement of the merging trigger.

Clause 100. The administration device of clause 99, wherein movement of the carriage relative to the storage assembly is linear.

Clause 101. The administration device of clause 100, wherein the cam surface and the cam follower comprise opposing inclined surfaces, relative rotation between the opposing inclined surfaces resulting in the linear movement of the carriage.

Clause 102. The administration device of clause 93, further comprising: a pressure chamber configured to receive a pressurized fluid, wherein the storage assembly is disposed within the pressure chamber and the activation system further comprises a pressurized fluid source arranged such that action of the activation system actuates the pressurized fluid source to provide a pressurized fluid to the pressure chamber to increase a pressure within the pressure chamber.

Clause 103. The administration device of clause 102, wherein the first interior volume comprises a first substance, the second interior volume comprises a second substance, and merging of the first interior volume and the second interior volume creates a reconstituted interior volume of the storage assembly contains a beneficial agent solution comprising the first substance and the second substance, wherein the increase in pressure acts on the reconstituted interior volume to expel the beneficial agent solution from the storage assembly.

Clause 104. The administration device of clause 102, wherein the merging trigger comprises an external portion that is external to the pressure chamber and an internal portion disposed within the pressure chamber, the internal portion further comprising a interface element of the merging mechanism configured to move relative to the storage assembly in response to the movement of the merging trigger.

Clause 105. The administration device of clause 102, further comprising: a sealed interface with the merging trigger to prevent or regulate flow of the pressurized fluid from the pressure chamber.

Clause 106. The administration device of clause 93, wherein the first interior volume comprises a first substance, the second interior volume comprises a second substance, and merging of the first interior volume and the second interior volume creates a reconstituted interior volume of the storage assembly contains a beneficial agent solution comprising the first substance and the second substance.

Clause 107. The administration device of clause 106, wherein the first substance comprises a diluent and the second substance comprises an active ingredient of the beneficial agent.

Clause 108. The administration device of clause 106, wherein the activation system, when in the armed state, is configured to allow activation of the administration device to expel the beneficial agent solution from the reconstituted interior volume through a dispensing element of the administration device.

Clause 109. The administration device of clause 93, the arming system further comprising: an arming trigger configured to, in response to manipulation of the arming trigger, initiate the transition of the activation system between the safe state and the armed state.

Clause 110. The administration device of clause 109, wherein the arming system is not acted upon by manipulation of the merging mechanism.

Clause 111. The administration device of clause 109, wherein actuation of the arming trigger is substantially prevented prior to manipulation of the merging trigger and is substantially not prevented after manipulation of the merging trigger.

Clause 112. The administration device of clause 93, wherein the merging trigger is manipulable by a first manipulation to conduct the merging of the first interior volume and the second interior volume, and the merging trigger is manipulable by a second manipulation to cause the arming system to transition the activation system from the safe state to the armed state.

Clause 113. The administration device of clause 93, wherein the compression element is configured for compression of at least one of the first compartment or the second compartment to defeat the fluid isolation element.

Clause 114. The administration device of clause 113, wherein the compression element comprises at least one of a cross-member, a rod, a panel, or a roller, configured to increase pressure in at least one of the first compartment or the second compartment to defeat the separation between them.

Clause 115. The administration device of clause 93, wherein the storage assembly comprises a flexible package, the fluid isolation element comprising a frangible seal disposed between the first interior volume and the second interior volume.

Clause 116. The administration device of clause 115, wherein the storage assembly includes a first wall and a second wall sealed against the first wall to define the first interior volume, the second interior volume, and the frangible seal.

Clause 117. The administration device of clause 115, wherein the storage assembly further comprises a port configured to communicate the beneficial agent from the storage assembly to a dispensing element of the administration device upon activation of the activation system.

Clause 118. The administration device of clause 115, wherein the storage assembly further comprises a third interior volume separate from the first interior volume and the second interior volume prior by a second fluid isolation element, the third interior volume containing a constituent of the beneficial agent different from contents of the first interior volume and the second interior volume, the contact of the compression element further configured to defeat the second fluid isolation element and merge the third interior volume with the first interior volume and the second interior volume.

Clause 119. The administration device of clause 93, wherein the merging trigger includes any one or more of a cap, knob, lever, or button.

Clause 120. The administration device of clause 93, wherein the merging trigger comprises at least a portion of a shell of the administration device.

Clause 121. The administration device of clause 93, further comprising an indicator that is configured to generate a signal responsive to manipulation of the merging trigger.

Clause 122. The administration device of clause 121, wherein the signal comprises at least one of a visual indication or a tactile indication.

Clause 123. The administration device of clause 122, wherein the signal conveys information related to a measurable value.

Clause 124. The administration device of clause 123, wherein the measurable value includes any one or more of a first elapsed time from merging, a second elapsed time from merging within a defined temperature range, or a mean kinetic temperature.

Clause 125. The administration device of clause 124, wherein the signal is generated by any one or more of a chemical process, a physical process, an enzymatic process, or a biological process.

Clause 126. The administration device of clause 121, wherein the signal includes a binary indication of usability or non-usability of a mixed substance comprising the beneficial agent.

Clause 127. The administration device of clause 121, wherein the activation system is rendered manipulable by the movement of the merging trigger.

Clause 128. The administration device of clause 93, wherein the movement of the merging trigger includes rotation or linear motion of the merging trigger.

Clause 129. The administration device of clause 93, wherein the arming system is configured to transition the activation system between the safe state and the armed state based on a rotation or linear motion of a first portion of the administration device relative to a second portion of the administration device.

Clause 130. The administration device of clause 93, wherein the merging trigger is manipulable by rotation or linear motion to activate the activation system in the armed state.

Clause 131. The administration device of clause 93, wherein the activation system is manipulable by rotation or linear motion to activate the activation system.

Unless otherwise indicated, all number or expressions, such as those expressing dimensions, physical characteristics, time etc., used in the specification (other than the claims) are understood as modified in all instances by the term “approximately”. At the very least, and not as an attempt to limit the application of the doctrine of equivalents to the claims, each numerical parameter recited in the specification or claims which is modified by the term “approximately” should at least be construed in light of the number of recited significant digits and by applying rounding techniques. Moreover, all ranges disclosed herein are to be understood to encompass and provide support for claims that recite any and all sub-ranges or any and all individual values subsumed therein. For example, a stated range of 1 to 10 should be considered to include and provide support for claims that recite any and all sub-ranges or individual values that are between and/or inclusive of the minimum value of 1 and the maximum value of 10; that is, all sub-ranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

Although the technology has been described in language that is specific to certain structures and materials, it is to be understood that the invention defined in the appended claims is not necessarily limited to the specific structures and materials described. Rather, the specific aspects are described as forms of implementing the claimed invention. Because many embodiments of the invention can be practiced without departing from the spirit and scope of the invention, the invention is not limited except as provided in the claims hereinafter appended.

Throughout this disclosure, the terms “proximal” and “distal” are used to orient the administration device and the configuration of the device. As used herein, the term “proximal” generally refers to a direction towards the base, or dispensing end of the administration device. As used herein, the term “distal” refers to a direction towards the end furthest from the base, or dispensing end of the administration device.

1. An administration device for administering a beneficial agent, comprising:
a storage assembly comprising a first compartment and a second compartment, the first compartment fluidly separated from the second compartment by a fluid isolation element that isolates a first interior volume of the first compartment from a second interior volume of the second compartment;
a merging mechanism, comprising:
a first interface element;
a merging trigger comprising a second interface element, the second interface element being engageable with the first interface element to move relative to the storage assembly in response to manipulation of the merging trigger; and
a compression element supportably engaged to the first interface element and moveable therewith, the compression element being disposed for contact of at least one of the first compartment or the second compartment in response to movement of the first interface element relative to the storage assembly to defeat the fluid isolation element and merge the first interior volume and the second interior volume; and
an arming system engageable with an activation system of the administration device, the arming system configured to transition the activation system between a safe state in which the activation system is configured to prevent activation of the administration device for dispensing the beneficial agent and an armed state in which the activation system is configured to allow activation of the administration device for dispensing the beneficial agent;
wherein the manipulation of the merging trigger is independent from action of the arming system.

2. The administration device of claim 1, the merging mechanism further comprising:
a carriage comprising the first interface element, the second interface element being engageable with the first interface element to move the carriage relative to the storage assembly in response to manipulation of the merging trigger;
wherein the compression element is supportably engaged by the carriage and moveable therewith, the compression element being disposed for contact of at least one of the first compartment or the second compartment in response to movement of the carriage relative to the storage assembly.

3. The administration device of claim 2, wherein one of the first interface element or the second interface element comprises a cam surface, the other of the first interface element or the second interface element comprises a cam follower, and wherein manipulation of the merging trigger results in relative movement of the cam surface and the cam follower to move the carriage relative to the storage assembly.

4. The administration device of claim 3, wherein the cam surface and the cam follower undergo the relative movement in response to rotational movement of the merging trigger.

5. The administration device of claim 4, wherein movement of the carriage relative to the storage assembly is linear movement.

6. The administration device of claim 5, wherein the cam surface and the cam follower comprise opposing inclined surfaces, relative rotation between the opposing inclined surfaces resulting in the linear movement of the carriage.

7. The administration device of claim 1, wherein the first interior volume comprises a first substance, the second interior volume comprises a second substance, and merging of the first interior volume and the second interior volume creates a reconstituted interior volume of the storage assembly contains a beneficial agent solution comprising the first substance and the second substance.

8. The administration device of claim 7, wherein the first substance comprises a diluent and the second substance comprises an active ingredient of the beneficial agent.

9. The administration device of claim 7, wherein the activation system, when in the armed state, is configured to allow activation of the administration device to expel the beneficial agent solution from the reconstituted interior volume through a dispensing element of the administration device.

10. The administration device of claim 7, further comprising:
a pressure chamber configured to receive a pressurized fluid, wherein the storage assembly is disposed within the pressure chamber; and
wherein the activation system further comprises a pressurized fluid source arranged such that action of the activation system actuates the pressurized fluid source to provide a pressurized fluid to the pressure chamber to increase a pressure within the pressure chamber;
wherein the increase in pressure acts on the reconstituted interior volume to expel the beneficial agent solution through a dispensing element of the administration device.

11. The administration device of claim 10, wherein the merging trigger comprises an external portion that is external to the pressure chamber and an internal portion disposed within the pressure chamber, the internal portion further comprising the second interface element.

12. The administration device of claim 10, further comprising:
a sealed interface with the merging trigger to prevent or regulate flow of the pressurized fluid from the pressure chamber.

13. The administration device of claim 1, further comprising:
an arming trigger configured to, in response to manipulation of the arming trigger, initiate the transition of the activation system between the safe state and the armed state.

14. The administration device of claim 13, wherein the arming system is not acted upon by manipulation of the merging mechanism.

15. The administration device of claim 13, wherein actuation of the arming trigger is substantially prevented prior to manipulation of the merging trigger and is substantially not prevented after manipulation of the merging trigger.

16. The administration device of claim 1, wherein the merging trigger is manipulable by a first manipulation to conduct the merging of the first interior volume and the second interior volume, and the merging trigger is manipulable by a second manipulation to cause the arming system to transition the activation system from the safe state to the armed state.

17. The administration device of claim 1, wherein the compression element is configured for compression of at least one of the first compartment or the second compartment to defeat the fluid isolation element.

18. The administration device of claim 17, wherein the compression element comprises at least one of a cross-member, a rod, a panel, or a roller, configured to increase pressure in at least one of the first or the second compartment to defeat the fluid isolation element.

19. The administration device of claim 1, wherein the storage assembly comprises a flexible package, the fluid isolation element comprising a frangible seal disposed between the first interior volume and the second interior volume.

20. The administration device of claim 19, wherein the storage assembly includes a first wall and a second wall sealed against the first wall to define the first interior volume, the second interior volume, and the frangible seal.

21-131. (canceled)
